(19) United States
(12) Patent Application Publication
(10) Pub. No.: US 2026/0315520 A1
(43) Pub. Date: Oct. 15, 2026

(54) IMPLANT POCKET IRRIGATION AND SILICONE REMOVAL SOLUTIONS AND METHODS

(71) Applicant: Stephen David Bresnick, Encino, CA (US)

(72) Inventor: Stephen David Bresnick, Encino, CA (US)

(21) Appl. No.: 19/643,189

(22) Filed: Apr. 9, 2026

Related U.S. Application Data
(60) Provisional application No. 63/786,078, filed on Apr. 9, 2025.

(51) Int. Cl. A61K 31/785; A61K 31/14; A61K 31/765; A61P 31/00

(52) U.S. Cl. CPC A61K 31/785; A61K 31/14; A61K 31/765; A61P 31/00

(57) ABSTRACT

Compositions and methods for irrigating surgically-created implant pockets in a subject, either prior to the insertion of a prosthesis implant or following the removal of an implant. The presently disclosed irrigation solutions may contain polyhexamethylene biguanide (PHMB), one or more poloxamer compounds, and water. In certain embodiments, the irrigation solutions may include poloxamer, either alone, or in combination with one or more additional poloxamer compounds. In certain embodiments, the irrigation solutions may further include benzalkonium chloride (BZK). The presently disclosed compositions and methods are operable to decrease the risk of infection and capsular contracture resulting from implant insertion and removal, as well as to remove silicone gel from an implant pocket resulting from the rupture of a silicone implant.

### CROSS-REFERENCE TO RELATED APPLICATIONS

This application claims priority to and the benefit of U.S. Provisional Application No. 63/786,078, entitled “Implant Pocket Irrigation and Silicone Removal Solutions and Methods,” filed on Apr. 9, 2025, the disclosure of which is incorporated herein by reference in its entirety for all purposes.

### FIELD OF TECHNOLOGY

The present disclosure is directed to compositions and methods for irrigating surgically-created implant pockets in a subject, either prior to the insertion of a prosthesis implant or following the removal of an implant. In certain embodiments, the presently disclosed compositions and methods are suitable for the irrigation and treatment of an implant pocket following the removal of a silicone implant, including a ruptured silicone implant. The presently disclosed compositions and methods are operable to decrease the risk of infection and capsular contracture following implant insertion or resulting from surgical insertion of prosthesis implants. Alternatively, the presently disclosed compositions and methods are operable to reduce the risk of granuloma formation, inflammation, infection, and systemic complications resulting from silicone gel leakage following the removal of ruptured silicone gel implants from the implant pocket. The presently disclosed irrigation solution compositions and methods are further useful to help remove silicone gel from an implant pocket resulting from the rupture of a silicone gel implant, thereby improving patient outcomes following the rupture of silicone gel implants. In certain embodiments, the presently disclosed irrigation solution compositions and methods may be used to irrigate dissected tissue and channels created therein during the surgical removal of injected silicone.

### BACKGROUND

A prosthetic implant, also known as simply an implant or a prosthesis, is an artificial device that may be inserted into the body of a subject for cometic purposes or to replace a missing body part, enhance a body part, or to help restore a body part's function. Common examples of prosthetic implants include breast implants, gluteal or buttock implants, facial implants, pacemakers, joint replacement prosthesis, and tissue grafts. The surgical insertion and removal of prosthetic implants often carries the risk of infection as well as several other potential complications.

In the specific case of cosmetic surgeries involving the insertion or removal of cosmetic implants, there is a continued interest in reducing the risk of both infection and capsular contracture. Capsular contracture refers to a complication where scar tissue forms around the implant, resulting in tightening and hardening of the capsule surrounding the implant which, in turn, may cause discomfort and/or suboptimal aesthetic effects, including a misshapen or unnatural appearing implant. In some cases, surgical revision may be required to remove or replace the implant as well as to remove the scar tissue or capsule surrounding the original implant, via a surgical procedure known as “capsulectomy.” It is believed that several factors may contribute to the incidence of capsular contracture, including infection arising out of implant insertion, the development of a biofilm surrounding the implant, and the post-insertion rupture of silicone implants resulting in silicone gel leakage and migration. Even in the absence of the post-insertion rupture of silicone implants, silicone may eventually bleed through the implant shell and enter the surrounding tissue, even if the implant shell remains intact. Silicone bleed without implant rupture can lead to various complications, symptoms, and undesirable patient outcomes. Irrespective of the manner by which the silicone gel contacts or invades surrounding tissues, when silicone gel comes into contact with tissue it elicits inflammation and other adverse patient outcomes.

Capsular contracture remains the most common complication of aesthetic breast augmentation despite advances in the understanding of the biological processes which appear to be involved. The use of antibiotic or antiseptic irrigation solutions as well as the use of implant delivery devices, skin barriers, and nipple shields has assisted in the reduction of capsular contracture. Yet, despite current practice improvements, a significant number of women develop capsular contracture following breast augmentation and require revisional surgery or live with discomfort, deformity, or suboptimal results. Accordingly, additional compositions and methods for reducing the risk of infection and other complications, such as capsular contracture, during prosthetic implant insertion and removal are desirable.

The use of silicone gel implants poses additional challenges. Common examples of silicone gel implants include silicone breast implants, silicone gluteal implants, silicone buttock implants, and silicone facial implants. Most silicone implants are expected to last at least twelve (12) to fifteen (15) years when properly manufactured and surgically inserted. However, the post-insertion rupture of silicone implants is a possible outcome with the probability of implant rupture increasing as the implant ages. When a silicone implant ruptures, the silicone gel contained within the silicone shell of the implant may escape the implant shell and invade into the fibrous capsule surrounding the implant (intracapsular) or may migrate beyond the fibrous capsule (extracapsular) to invade the surrounding tissues, often producing scar tissue and inflammation. Substantial or complete removal of the silicone that has leaked from the silicone implant is desirable in order to mitigate or prevent additional complications. However, silicone gel adheres to implant pocket tissues and removal of the gel from the implant pocket is often difficult, labor-intensive, and time-consuming. Accordingly, additional compositions and methods for removing silicone gel from the implant pocket of a subject are desirable.

### SUMMARY

To address these shortcomings, the applicant has discovered new compositions and methods for irrigating surgically-created implant pockets in a subject, either prior to the insertion of a prosthesis implant or following the removal of an implant. The presently disclosed compositions, also referred to in this disclosure as “irrigation solutions” or “silicone removal solutions,” are aqueous solutions comprising polyhexamethylene biguanide (PHMB), one or more poloxamer compounds, and water. In certain embodiments, the irrigation solutions may include poloxamer, PHMB, and water. In other embodiments, the presently disclosed irrigation solutions may include a mixture of poloxamer 188 and one or more additional poloxamer compounds. In certain embodiments, each of the one or more poloxamer compounds in the presently disclosed irrigations solutions are characterized by an ethylene oxide (EO) content of at least 70%. According to certain aspects of the present disclosure, the irrigation solutions may further include benzalkonium chloride (BZK). The presently disclosed compositions and methods are operable to decrease the risk of infection and capsular contracture resulting from implant insertion and removal, as well as mitigate or reduce the complications coincident with surgical insertion or removal of prosthetic implants.

In certain embodiments, the presently disclosed compositions and methods are especially suited for the irrigation and treatment of an implant pocket following the removal of a silicone implant, including a ruptured silicone implant. In particular, the presently disclosed compositions and methods are operable to reduce the risk of granuloma formation, inflammation, infection, and systemic complications resulting from silicone gel leakage following the removal of a ruptured silicone gel implant from an implant pocket. The presently disclosed irrigation solution compositions and methods are further effective to facilitate removal of silicone gel from an implant pocket resulting from the rupture of a silicone gel implant or as a result of silicone bleed, thereby improving patient outcomes for patients suffering from a ruptured silicone gel implant. In certain embodiments, the presently disclosed irrigation solution compositions and methods may be used to irrigate dissected tissue and channels created therein during the surgical removal of injected silicone.

Still other aspects and advantages of these exemplary embodiments and other embodiments, are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

In order to describe the manner in which the advantages and features of the disclosure can be obtained, reference is made to embodiments thereof which are illustrated in the appended drawings. One of skill in the art will understand that these drawings depict only exemplary embodiments of the disclosure and are not therefore to be considered to be limiting of its scope, the principles herein are described and explained with additional specificity and detail through the use of the accompanying drawings in which:

FIG. 1 is a diagrammatic representation of an example method for irrigation of a surgically-created implant pocket in a subject, according to an exemplary embodiment of the present disclosure;

FIG. 2 is a diagrammatic representation of an example alternative method for irrigating a surgically-created implant pocket in a subject, according to an exemplary embodiment of the present disclosure;

FIG. 3 is a diagrammatic representation of an example method for irrigating an implant pocket in a subject during surgical insertion of a prosthetic implant into a subject, according to an exemplary embodiment of the present disclosure;

FIGS. 4A-4B is a diagrammatic representation of an example method for treating an implant pocket or removing silicone from an implant pocket during the removal or replacement of a silicone gel implant in a subject, according to an exemplary embodiment of the present disclosure;

FIGS. 5A-5B is a diagrammatic representation of an example method of removing or replacing a ruptured silicone gel implant in a subject in need thereof, according to an exemplary embodiment of the present disclosure; and

FIG. 6 is a diagrammatic representation of a method of treating or irrigating a surgically-created implant pocket in a subject, according to an exemplary embodiment of the present disclosure.

### DETAILED DESCRIPTION

The present disclosure provides various embodiments related to compositions and methods for irrigating surgically-created implant pockets in a subject, either prior to the insertion of a prosthesis implant or following the removal of an implant. Further embodiments may be described and disclosed.

In the following description, numerous details are set forth in order to provide a thorough understanding of the various embodiments. In other instances, well-known processes, devices, and systems may not have been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “reducing,” “reduced,” or any variation thereof, when used in the claims and/or the specification includes any measurable decrease or complete inhibition to achieve a desired result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

It will be appreciated that numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

The present disclosure provides new compositions and methods for irrigating surgically-created implant pockets in a subject. The compositions and methods may be used to irrigate or rinse an implant pocket in a subject either before a prosthetic implant is surgically inserted into the implant pocket or following the surgical removal of the implant from the implant pocket. The implant pocket may be created by a surgeon during the surgical insertion of an implant in a subject. In particular, during implant insertion, a surgeon may create or otherwise cause an incision in the skin of the subject and then proceeds to create or dissect an implant pocket into which the implant may be inserted. Therefore, the surgically-created implant pocket is a cavity in the subject that contains or is intended to contain a prosthetic implant. Because the implant pocket is generally located well beneath the incision in the skin of a subject, the surgeon may also generate a dissection tunnel leading from the incision to the implant pocket. Therefore, during implant insertion, the implant may pass through the dissection tunnel that connects the incision to the implant pocket.

The presently disclosed compositions for irrigating a surgically-created implant pocket in a subject, also referred to herein variously as “irrigation solutions” or “silicone removal solutions,” may be operable to decrease the risk of infection and capsular contracture resulting from implant insertion and removal, as well as mitigate or reduce the complications coincident with surgical insertion or removal of prosthetic implants. In certain embodiments, the presently disclosed compositions and methods are especially suited for the irrigation and treatment of an implant pocket following the removal of a silicone implant, including a ruptured silicone implant. In particular, the presently disclosed compositions and methods are operable to reduce the risk of granuloma formation, inflammation, infection, and systemic complications resulting from silicone gel leakage following the removal of a ruptured silicone gel implant from an implant pocket. The presently disclosed irrigation solution compositions and methods are further effective to facilitate removal of silicone gel from an implant pocket resulting from the rupture of a silicone gel implant, thereby improving patient outcomes for patients suffering from a ruptured silicone gel implant.

The presently disclosed compositions may include one or more poloxamer compounds. The Applicant has surprisingly discovered that compositions comprising one or more poloxamer compounds, including, for example, poloxamer 188 is effective for loosening, liberating, or removing silicone gel from tissues in need of treatment and/or is useful to facilitate debridement or removal of tissue debris that comprises silicone gel. The Applicant has further unexpectedly discovered that compositions comprising one or more poloxamer compounds may be effective for the removal of silicone from such tissues even at concentrations not previously reported for effective use in other uses, such as in surface or external cleansing applications.

Poloxamer compounds, also known as “poloxamers,” are nonionic triblock copolymers composed of a central hydrophobic chain of polyoxypropylene (poly(propylene oxide) that is flanked by two hydrophilic chains of polyoxyethylene (poly (ethylene oxide). A non-limiting list of exemplary poloxamer compounds as well as their representative properties is provided in Table 1.

TABLE 1

| | Ethylene Oxide (EO) | Average Molecular | Viscosity |
|---|---|---|---|
| Poloxamer | Content (%) | Weight | (Pa · s) |
| P105 | 50 | 1900 | 0.375 |
| P108 | 80 | 4700 | 0.260 |
| P122 | 20 | 1630 | 0.280 |
| P123 | 30 | 1850 | 0.310 |
| P124 | 40 | 2200 | 0.440 |
| P182 | 20 | 2500 | 0.450 |
| P183 | 30 | 2650 | 0.490 |
| P184 | 40 | 2900 | 0.850 |
| P185 | 50 | 3400 | 0.180 |
| P188 | 80 | 8400 | 1.000 |
| P212 | 20 | 2750 | 0.510 |
| P215 | 50 | 4150 | 0.250 |
| P217 | 70 | 6600 | 0.480 |
| P234 | 40 | 4200 | 0.280 |
| P235 | 50 | 4600 | 0.310 |
| P237 | 70 | 7700 | 0.700 |
| P238 | 80 | 11400 | 2.300 |
| P288 | 80 | 13000 | 2.700 |
| P333 | 30 | 4950 | 0.285 |
| P334 | 40 | 5900 | 0.390 |
| P335 | 50 | 6500 | 0.750 |
| P338 | 80 | 14600 | 2.800 |
| P402 | 20 | 5000 | 1.750 |
| P403 | 30 | 5750 | 0.350 |
| P407 | 70 | 12600 | 3.100 |

In certain embodiments, the one or more poloxamer compounds may be selected from poloxamer 188, poloxamer 407, poloxamer 108, poloxamer 1107, poloxamer 338, poloxamer 101, poloxamer 105, poloxamer 122, poloxamer 123, poloxamer 124, poloxamer 181, poloxamer 182, poloxamer 183, poloxamer 184, poloxamer 185, poloxamer 212, poloxamer 215, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 401, poloxamer 402, poloxamer 403, poloxamer 105 benzoate, poloxamer 182 dibenzoate, and any combination thereof. In other embodiments, the one or more poloxamer compounds may be selected from poloxamer 188, poloxamer 407, poloxamer 108, poloxamer 1107, poloxamer 338, and any combination thereof. In still other embodiments, the one or more poloxamer compound may be selected from poloxamer 188, poloxamer 407, poloxamer 108, poloxamer 338, poloxamer 217, poloxamer 237, poloxamer 238, poloxamer 288, and any combination thereof. In certain embodiments, the one or more poloxamer compounds may each be characterized by an ethylene oxide (EO) content of at least 70%. For example, in certain embodiments, the one or more poloxamer compounds may be selected from poloxamer compounds that are characterized by an ethylene oxide (EO) content of at least 70%.

In certain embodiments, the presently disclosed compositions may include poloxamer 188 as the only poloxamer compound present in the composition. In other embodiments, poloxamer 407, or poloxamer 108, or poloxamer 338, or poloxamer 217, or poloxamer 237, or poloxamer 238, or poloxamer 288 may be the only poloxamer compound present in the presently disclosed composition.

In certain embodiments, the one or more poloxamer compounds may be a mixture of poloxamer 188 and one or more additional poloxamer compounds. For example, the one or more poloxamer compounds may be a mixture of poloxamer 188 and poloxamer 407. In other cases, the one or more poloxamer compounds may be a mixture of poloxamer 188, poloxamer 407, and poloxamer 1107. In some embodiments, the one or more additional poloxamer compounds may be selected from the group consisting of poloxamer 407, poloxamer 108, poloxamer 338, poloxamer 217, poloxamer 237, poloxamer 238, poloxamer 288, and combinations thereof.

In certain embodiments, the presently disclosed compositions may include two or more poloxamer compounds. For example, the presently disclosed compositions may include two or more poloxamer compounds selected from the group consisting of poloxamer 188, poloxamer 407, poloxamer 108, poloxamer 338, poloxamer 217, poloxamer 237, poloxamer 238, poloxamer 288, poloxamer 1107, and any combination thereof. In other embodiments, the presently disclosed compositions may include two or more poloxamer compounds selected from the group consisting of poloxamer 188, poloxamer 407, poloxamer 108, poloxamer 1107, poloxamer 338, and any combination thereof. In certain embodiments, the two or more poloxamer compounds may include poloxamer 188.

The amount or concentration of the one or more poloxamer compounds used in the presently disclosed irrigation solutions and silicone removal solutions may depend on the specific surgical use scenario and anatomical location in which irrigation solution or silicone removal solution is used. For example, in situations in which there is a relatively large amount of silicone gel present in an implant pocket, or the silicone gel is particularly difficult to remove, or has significantly penetrated the contacted tissue, compositions having a higher concentration of one or more poloxamers may be selected for use or treatment of the locus in need of treatment. In other cases, for example, when surgical removal of the vast majority of silicone gel has been possible or successful, or there is minimal invasion of the contacted tissues by the silicone gel, or the silicone gel is relatively easily liberated from the tissue, compositions having a lower concentration of one or more poloxamers may be selected for use or treatment of the locus in need of treatment.

In certain embodiments, the one or more poloxamer compounds may comprise from about 0.5 weight % to about 65 weight % of the total weight of the composition. In other embodiments, the one or more poloxamer compounds may comprise from about 0.5 weight % to about 55 weight %, or from about 0.5 weight % to about 50 weight %, or from about 5 weight % to about 65 weight %, or from about 10 weight % to about 65 weight %, or from about 15 weight % to about 65 weight %, or from about 20 weight % to about 65 weight %, or from about 25 weight % to about 65 weight %, or from about 25 weight % to about 55 weight %, or from about 25 weight % to about 50 weight %, or from about 25 weight % to about 45 weight %, or from about 25 weight % to about 40 weight %, or from about 25 weight % to about 35 weight %, or from about 15 weight % to about 50 weight %, of the total weight of the composition.

In other embodiments, the one or more poloxamer compounds may comprise from about 5.0 weight % to about 18 weight %, or from about 5.0 weight % to about 15 weight %, or from 5.0 weight % to about 12 weight %, or from about 5.0 weight % to about 10 weight %, or from 5.0 weight % to about 7.5 weight %, or from about 7.5 weight % to about 18 weight %, or from about 7.5 weight % to about 15 weight %, or from about 7.5 weight % to about 12 weight %, or from about 7.5 weight % to about 10 weight %, based on the total weight of the composition.

In still other embodiments, the one or more poloxamer compounds may comprise from about 0.5 weight % to about 7.5 weight %, or from about 0.5 weight % to about 10 weight %, or from about 0.5 weight % to about 12 weight %, or from about 0.5 weight % to about 15 weight %, or from about 0.5 weight % to about 18 weight %, or from about 2.5 weight % to about 7.5 weight %, or from about 2.5 weight % to about 10 weight %, or from about 2.5 weight % to about 12 weight %, or from about 2.5 weight % to about 15 weight %, or from about 2.5 weight % to about 18 weight %, or from about 3.5 weight % to about 7.5 weight %, or from about 3.5 weight % to about 10 weight %, or from about 3.5 weight % to about 12 weight %, or from about 3.5 weight % to about 15 weight %, or from about 3.5 weight % to about 18 weight %, or from about 4.5 weight % to about 7.5 weight %, or from about 4.5 weight % to about 10 weight %, or from about 4.5 weight % to about 12 weight %, or from about 4.5 weight % to about 15 weight %, or from about 4.5 weight % to about 18 weight %, based on the total weight of the composition.

In other embodiments, the one or more poloxamer compounds may comprise from about 0.5 weight % to about 5.0 weight % of the total weight of the composition. In other embodiments, the one or more poloxamer compounds may comprise from about 1.0 weight % to about 4.0 weight % of the total weight of the composition. In still other embodiments, the one or more poloxamer compounds may comprise from about 2.0 weight % to about 5.0 weight % of the total weight of the composition.

In certain embodiments, the presently disclosed compositions may include two or more poloxamer compounds, or three or more poloxamer compounds, or four or more poloxamer compounds. In such instances, the plurality of poloxamer compounds included in the presently disclosed compositions may have a combined weight according to the presently disclosed weight % ranges for the one or more poloxamer compounds. For example, the combined weight of the one or more poloxamer compounds or the combined weight of the plurality of poloxamer compounds may be from about 0.5 weight % to about 65 weight %, or from about 0.5 weight % to about 55 weight %, or from about 0.5 weight % to about 50 weight %, or from about 5 weight % to about 65 weight %, or from about 10 weight % to about 65 weight %, or from about 15 weight % to about 65 weight %, or from about 20 weight % to about 65 weight %, or from about 25 weight % to about 65 weight %, or from about 25 weight % to about 55 weight %, or from about 25 weight % to about 50 weight %, or from about 25 weight % to about 45 weight %, or from about 25 weight % to about 40 weight %, or from about 25 weight % to about 35 weight %, or from about 15 weight % to about 50 weight %, or from about 5.0 weight % to about 18 weight %, or from about 5.0 weight % to about 15 weight %, or from 5.0 weight % to about 12 weight %, or from about 5.0 weight % to about 10 weight %, or from 5.0 weight % to about 7.5 weight %, or from about 7.5 weight % to about 18 weight %, or from about 7.5 weight % to about 15 weight %, or from about 7.5 weight % to about 12 weight %, or from about 7.5 weight % to about 10 weight %, or from about 0.5 weight % to about 7.5 weight %, or from about 0.5 weight % to about 10 weight %, or from about 0.5 weight % to about 12 weight %, or from about 0.5 weight % to about 15 weight %, or from about 0.5 weight % to about 18 weight %, or from about 2.5 weight % to about 7.5 weight %, or from about 2.5 weight % to about 10 weight %, or from about 2.5 weight % to about 12 weight %, or from about 2.5 weight % to about 15 weight %, or from about 2.5 weight % to about 18 weight %, or from about 3.5 weight % to about 7.5 weight %, or from about 3.5 weight % to about 10 weight %, or from about 3.5 weight % to about 12 weight %, or from about 3.5 weight % to about 15 weight %, or from about 3.5 weight % to about 18 weight %, or from about 4.5 weight % to about 7.5 weight %, or from about 4.5 weight % to about 10 weight %, or from about 4.5 weight % to about 12 weight %, or from about 4.5 weight % to about 15 weight %, or from about 4.5 weight % to about 18 weight %, based on the total weight of the composition.

In certain embodiments, the combined weight of the one or more poloxamer compounds in the presently disclosed compositions may be less than 2 weight %, or less than 20 weight %, or less than 18 weight %, or less than 15 weight %, or less than 12 weight %, or less than 10 weight %, or less than 7.5 weight, or less than 5 weight %, based on the total weight of the composition. In other embodiments, the combined weight of the one or more poloxamer compounds in the presently disclosed compositions may be greater than 24 weight %, or greater than 25 weight %, or greater than 28 weight %, or greater than 30 weight %, or greater than 35 weight %, or greater than 4 weight %, or greater than 5 weight %, or greater than 7.5 weight %, based on the total weight of the composition. In other embodiments, the combined weight of the one or more poloxamer compounds in the presently disclosed compositions may be less than about 1.5 weight %, or less than about 1.25 weight %, or less than about 1.0 weight %, based on the total weight of the composition. In certain embodiments, the weight of each of the one or more poloxamer compounds in the presently disclosed compositions may be less than about 2.0 weight %, or less than about 1.5 weight %, or less than about 1.0 weight %, based on the total weight of the composition. In other embodiments, each of the one or more poloxamer compounds may be present in the presently disclosed compositions in an amount of from about 0.4 weight % to about 0.8 weight %, or from about 0.4 weight percent to about 0.6 weight %, based on the total weight of the composition. In other embodiments, each of the one or more poloxamer compounds is present in the presently disclosed compositions in an amount of about 0.5 weight %, based on the total weight of the composition.

In certain embodiments, poloxamer 188 may comprise from about 0.5 weight % to about 5.0 weight % of the total weight of the composition. In other embodiments, poloxamer 188 may comprise from about 1.0 weight % to about 4.0 weight % of the total weight of the composition. In still other embodiments, poloxamer 188 may comprise from about 2.0 weight % to about 5.0 weight % of the total weight of the composition.

In certain embodiments, poloxamer 188 may comprise less than about 2.0 weight %, or less than about 1.5 weight %, or less than about 1.0 weight % of the presently disclosed compositions, based on the total weight of the composition. In other embodiments, poloxamer 188 may be present in the presently disclosed compositions in an amount of from about 0.4 weight % to about 0.8 weight %, or from about 0.4 weight percent to about 0.6 weight %, based on the total weight of the composition. In other embodiments, poloxamer 188 may be present in the presently disclosed compositions in an amount of about 0.5 weight %, based on the total weight of the composition. In still other embodiments, poloxamer 188 may comprise from about 0.5 weight % to about 65 weight %, or from about 0.5 weight % to about 55 weight %, or from about 0.5 weight % to about 50 weight %, or from about 5 weight % to about 65 weight %, or from about 10 weight % to about 65 weight %, or from about 15 weight % to about 65 weight %, or from about 20 weight % to about 65 weight %, or from about 25 weight % to about 65 weight %, or from about 25 weight % to about 55 weight %, or from about 25 weight % to about 50 weight %, or from about 25 weight % to about 45 weight %, or from about 25 weight % to about 40 weight %, or from about 25 weight % to about 35 weight %, or from about 15 weight % to about 50 weight %, or from about 5.0 weight % to about 18 weight %, or from about 5.0 weight % to about 15 weight %, or from 5.0 weight % to about 12 weight %, or from about 5.0 weight % to about 10 weight %, or from 5.0 weight % to about 7.5 weight %, or from about 7.5 weight % to about 18 weight %, or from about 7.5 weight % to about 15 weight %, or from about 7.5 weight % to about 12 weight %, or from about 7.5 weight % to about 10 weight %, or from about 0.5 weight % to about 7.5 weight %, or from about 0.5 weight % to about 10 weight %, or from about 0.5 weight % to about 12 weight %, or from about 0.5 weight % to about 15 weight %, or from about 0.5 weight % to about 18 weight %, or from about 2.5 weight % to about 7.5 weight %, or from about 2.5 weight % to about 10 weight %, or from about 2.5 weight % to about 12 weight %, or from about 2.5 weight % to about 15 weight %, or from about 2.5 weight % to about 18 weight %, or from about 3.5 weight % to about 7.5 weight %, or from about 3.5 weight % to about 10 weight %, or from about 3.5 weight % to about 12 weight %, or from about 3.5 weight % to about 15 weight %, or from about 3.5 weight % to about 18 weight %, or from about 4.5 weight % to about 7.5 weight %, or from about 4.5 weight % to about 10 weight %, or from about 4.5 weight % to about 12 weight %, or from about 4.5 weight % to about 15 weight %, or from about 4.5 weight % to about 18 weight %, based on the total weight of the composition.

The Applicant has surprisingly discovered that the presently disclosed compositions are both safe and effective in facilitating silicone gel removal from the implant pocket and/or reducing infection, biofilm development, and capsular contracture rates, even when the concentration of the one or more poloxamer compounds is greater than that typically used in antiseptic and/or wound cleansing solutions. Implant pocket irrigation compositions containing relatively high weight % concentrations of one or more poloxamer compounds may be particularly appropriate, for example, when large amounts of residual or difficult to remove silicone gel is present in the implant pocket. Additionally, the Applicant has surprisingly discovered that the compositions containing relatively high weight % concentrations of one or more poloxamer compounds may be more effective at facilitating silicone gel removal from the implant pocket and/or reducing infection, biofilm development, and capsular contracture rates when combined with PHMB and/or the one or more poloxamer compounds is effective at higher concentrations or weight % (e.g., greater than 25 weight %) when combined with PHMB. In certain embodiments, synergistic results for compositions containing greater than 25 weight % one or more poloxamer compounds and PHMB may be observed.

The presently disclosed compositions may also include polyhexamethylene biguanide (PHMB), also known as polihexanide or polyaminopropyl biguanide. PHMB is a polymeric antiseptic compound and disinfectant. Surprisingly, it has been discovered by the Applicant that PHMB is effective in helping to loosen or liberate silicone gel from tissues within the implant pocket and/or facilitate debridement or removal of tissue debris that comprises silicone gel. The applicant has also unexpectedly discovered that the combination of PHMB and one or more poloxamer compounds produces synergistic results in which the removal of silicone gel, decrease in infection rates, decrease in capsular contracture rates, or improvement in patient outcomes is greater when delivered together according to the presently disclosed compositions and methods, than the sum of the effects observed when each compound is delivered independently. In certain embodiments, PHMB may be an optional component of the presently disclosed compositions and methods.

In certain embodiments, PHMB may comprise from about 0.01 weight % to about 0.5 weight % of the total weight of the composition. In other embodiments, PHMB may comprise from about 0.05 weight % to about 0.15 weight % of the total weight of the composition. In still other embodiments, PHMB may comprise from about 0.04 weight % to about 0.08 weight % of the total weight of the composition. In some embodiments, PHMB may comprise from about 0.04 weight % to about 0.06 weight % of the total weight of the composition. In certain embodiments, PHMB may comprise about 0.1 weight % of the total weight of the composition.

It has been unexpectedly discovered by the Applicant that PHMB may be effective in facilitating silicone gel removal from the implant pocket even at lower concentrations than typically used for antiseptic purposes. In particular, the Applicant has surprisingly discovered that PHMB may be effective in facilitating silicone gel removal at a concentration of less than 0.1 weight %, based on the total weight of the composition. More particularly, the Applicant has surprisingly discovered that PHMB may be effective in facilitating silicone gel removal at a concentration of from about 0.04 weight % to about 0.08 weight %, or more particularly from about 0.04 weight % to about 0.06 weight %, based on the total weight of the composition.

Additionally, the Applicant has unexpectedly discovered that the use of PHMB according to the presently disclosed irrigation compositions and methods may be effective in reducing the risk of infection, biofilm development, and capsular contracture in subjects at lower PHMB concentrations than typically used for antiseptic purposes. In particular, the Applicant has surprisingly discovered that PHMB may be effective in reducing infection, biofilm development, and capsular contracture rates at a concentration of less than 0.1 weight %, based on the total weight of the composition. More particularly, the Applicant has surprisingly discovered that PHMB may be effective in reducing infection, biofilm development, and capsular contracture rates at concentrations ranging from about 0.04 weight % to about 0.08 weight %, or more particularly from about 0.04 weight % to about 0.06 weight %, based on the total weight of the composition.

The Applicant has surprisingly discovered that the presently disclosed compositions are effective in facilitating silicone gel removal from the implant pocket and/or reducing infection, biofilm development, and capsular contracture rates, even when the concentration of the one or more poloxamer compounds is below that typically used in antiseptic and/or wound cleansing solutions. In particular, the Applicant has surprisingly discovered that the one or more poloxamer compounds may be more effective at facilitating silicone gel removal from the implant pocket and/or reducing infection, biofilm development, and capsular contracture rates when combined with PHMB and/or the one or more poloxamer compounds is effective at lower concentrations or weight % when combined with PHMB.

In particular, the Applicant has unexpectedly discovered that the presently disclosed compositions may be effective to facilitate silicone gel removal from the implant pocket and/or reduce infection, biofilm development, and capsular contracture rates, when the combined concentration of the one or more poloxamer compounds comprises less than 2 weight percent of the total weight of the composition. More particularly, the Applicant has unexpectedly discovered that the presently disclosed compositions may be effective to facilitate silicone gel removal from the implant pocket and/or reduce infection, biofilm development, and capsular contracture rates, when the combined concentration of the one or more poloxamer compounds comprises less than about 1.5 weight percent, or less than about 1.25 weight percent, or less than about 1.0 weight percent of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the concentration of each of the one or more poloxamer compounds is less than about 1.5 weight %, or about 1.0 weight percent or less, or from about 0.4 weight % to about 0.8 weight %, or from about 0.4 weight percent to about 0.6 weight %, or about 0.5 weight % of the total weight of the composition.

According to another aspect of the present disclosure, the Applicant has surprisingly discovered that the use of poloxamer 188, according to the presently disclosed compositions and methods, may be effective in facilitating silicone gel removal from the implant pocket and/or reducing infection, biofilm development, and capsular contracture rates, even at poloxamer 188 concentrations that are below that typically used in antiseptic and/or wound cleansing solutions. In particular, the Applicant has surprisingly discovered that poloxamer 188 is more effective at facilitating silicone gel removal from the implant pocket and/or reducing infection, biofilm development, and capsular contracture rates when combined with PHMB and/or poloxamer 188 is effective at lower concentrations or weight % when combined with PHMB.

In particular, the Applicant has unexpectedly discovered that the presently disclosed compositions containing poloxamer 188 may be effective to facilitate silicone gel removal from the implant pocket and/or reduce infection, biofilm development, and capsular contracture rates, when the poloxamer 188 concentration comprises less than 2 weight percent of the total weight of the composition. More particularly, the Applicant has unexpectedly discovered that the presently disclosed compositions may be effective to facilitate silicone gel removal from the implant pocket and/or reduce infection, biofilm development, and capsular contracture rates, when the poloxamer 188 concentration comprises less than about 1.5 weight percent, or less than about 1.25 weight percent, or less than about 1.0 weight percent of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the poloxamer 188 concentration is less than about 1.5 weight %, or about 1.0 weight percent or less, or from about 0.4 weight % to about 0.8 weight %, or from about 0.4 weight percent to about 0.6 weight %, or is about 0.5 weight % of the total weight of the composition.

According to another aspect of the present disclosure, the Applicant has surprisingly discovered that the presently disclosed compositions are effective in facilitating silicone gel removal from the implant pocket and/or reducing infection, biofilm development, and capsular contracture rates, even when the concentration of both PHMB and the one or more poloxamer compounds is below that typically used in antiseptic and/or wound cleansing solutions. In particular, the Applicant has unexpectedly discovered that the presently disclosed compositions may be effective to facilitate silicone gel removal from the implant pocket and/or reduce infection, biofilm development, and capsular contracture rates, when the combined weight % of PHMB and the one or more poloxamer compounds comprises less than 2 weight percent of the total weight of the composition. More particularly, the Applicant has unexpectedly discovered that the presently disclosed compositions may be effective to facilitate silicone gel removal from the implant pocket and/or reduce infection, biofilm development, and capsular contracture rates, when the combined weight % of PHMB and the one or more poloxamer compounds comprises less than about 1.5 weight percent, or less than about 1.25 weight percent, or less than about 1.0 weight percent of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the combined weight % of PHMB and the one or more poloxamer compounds is from about 0.4 weight % to about 0.8 weight %, or from about 0.4 weight percent to about 0.6 weight %, or about 0.5 weight % of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the weight % of PHMB is less than 0.1 weight %, or from about 0.04 weight % to about 0.08 weight %, or more particularly from about 0.04 weight % to about 0.06 weight %, and the one or more poloxamer compounds is less than about 2.0 weight %, or less than about 1.5 weight %, or less than about 1.0 weight %, or from about 0.4 weight % to about 0.8 weight %, or from about 0.4 weight percent to about 0.6 weight %, based on the total weight of the composition. In other embodiments, the Applicant has unexpectedly discovered that the presently disclosed compositions may be effective to facilitate silicone gel removal from the implant pocket and/or reduce infection, biofilm development, and capsular contracture rates, when the weight % of PHMB is less than 0.1 weight %, or from about 0.04 weight % to about 0.08 weight %, or more particularly from about 0.04 weight % to about 0.06 weight %, and the composition includes poloxamer 188 in an amount less than about 2.0 weight %, or less than about 1.5 weight %, or less than about 1.0 weight %, or from about 0.4 weight % to about 0.8 weight %, or from about 0.4 weight percent to about 0.6 weight %, based on the total weight of the composition.

According to certain aspects of the present disclosure, the presently disclosed compositions may further include benzalkonium chloride (BZK), also known as alkyldimethylbenzylammonium chloride. BZK is a cationic antiseptic and disinfectant. The Applicant has surprisingly discovered that BZK is particularly effective in helping to loosen or liberate silicone gel from tissues within the implant pocket and/or facilitate debridement or removal of tissue debris that comprises silicone gel. The applicant has also unexpectedly discovered that the combination of BZK with PHMB and/or one or more poloxamer compounds, according to the presently disclosed compositions and methods, produces synergistic results in which the removal of silicone gel, decrease in infection rates, decrease in capsular contracture rates, or improvement in patient outcomes is greater when delivered together according to the presently disclosed compositions and methods, than the sum of the effects observed when each compound is delivered independently.

In certain embodiments, BZK may comprise from about 0.01 weight % to about 0.5 weight % of the total weight of the composition. In other embodiments, BZK may comprise from about 0.05 weight % to about 0.15 weight % of the total weight of the composition. In still other embodiments, BZK may comprise from about 0.04 weight % to about 0.08 weight % of the total weight of the composition. In some embodiments, BZK may comprise from about 0.04 weight % to about 0.06 weight % of the total weight of the composition. In certain embodiments, BZK may comprise about 0.1 weight % or about 0.13 weight % of the total weight of the composition.

It has been unexpectedly discovered by the Applicant that BZK may be effective in facilitating silicone gel removal from the implant pocket even at lower concentrations than typically used for antiseptic purposes. In particular, the Applicant has surprisingly discovered that BZK may be effective in facilitating silicone gel removal at a concentration of less than about 0.13 weight % or less than about 0.10 weight %, based on the total weight of the composition. More particularly, the Applicant has surprisingly discovered that BZK may be effective in facilitating silicone gel removal at a concentration of from about 0.04 weight % to about 0.08 weight %, or more particularly from about 0.04 weight % to about 0.06 weight %, based on the total weight of the composition.

Additionally, the Applicant has unexpectedly discovered that the use of BZK according to the presently disclosed irrigation compositions and methods may be effective in reducing the risk of infection, biofilm development, and capsular contracture in subjects at lower BZK concentrations than typically used for antiseptic purposes. In particular, the Applicant has surprisingly discovered that BZK may be effective in reducing infection, biofilm development, and capsular contracture rates at a concentration of less than about 0.13 weight % or less than about 0.10 weight %, based on the total weight of the composition. More particularly, the Applicant has surprisingly discovered that BZK may be effective in reducing infection, biofilm development, and capsular contracture rates at concentrations ranging from about 0.04 weight % to about 0.08 weight %, or more particularly from about 0.04 weight % to about 0.06 weight %, based on the total weight of the composition.

According to another aspect of the present disclosure, the Applicant has surprisingly discovered that the presently disclosed compositions are effective in facilitating silicone gel removal from the implant pocket and/or reducing infection, biofilm development, and capsular contracture rates, even when the concentration of BZK, PHMB, and the one or more poloxamer compounds are each below that typically used in antiseptic and/or wound cleansing solutions. In particular, the Applicant has unexpectedly discovered that the presently disclosed compositions may be effective to facilitate silicone gel removal from the implant pocket and/or reduce infection, biofilm development, and capsular contracture rates, when the combined weight % of BZK, PHMB, and the one or more poloxamer compounds comprises less than 2 weight percent of the total weight of the composition. More particularly, the Applicant has unexpectedly discovered that the presently disclosed compositions may be effective to facilitate silicone gel removal from the implant pocket and/or reduce infection, biofilm development, and capsular contracture rates, when the combined weight % of BZK, PHMB, and the one or more poloxamer compounds comprises less than about 1.5 weight percent, or less than about 1.25 weight percent, or less than about 1.0 weight percent of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the combined weight % of BZK, PHMB, and the one or more poloxamer compounds is from about 0.4 weight % to about 0.8 weight %, or from about 0.4 weight percent to about 0.6 weight %, or about 0.5 weight % of the total weight of the composition. According to other aspects of the present disclosure, the Applicant has unexpectedly discovered that the presently disclosed compositions may be effective to facilitate silicone gel removal from the implant pocket and/or reduce infection, biofilm development, and capsular contracture rates, when the weight % of BZK and/or PHMB is each less than about 0.1 weight %, or from about 0.04 weight % to about 0.08 weight %, or more particularly from about 0.04 weight % to about 0.06 weight %, and the one or more poloxamer compounds is less than about 2.0 weight %, or less than about 1.5 weight %, or less than about 1.0 weight %, or from about 0.4 weight % to about 0.8 weight %, or from about 0.4 weight percent to about 0.6 weight %, based on the total weight of the composition. In other embodiments, the Applicant has unexpectedly discovered that the presently disclosed compositions may be effective to facilitate silicone gel removal from the implant pocket and/or reduce infection, biofilm development, and capsular contracture rates, when the weight % of BZK and/or PHMB is each less than about 0.1 weight %, or from about 0.04 weight % to about 0.08 weight %, or more particularly from about 0.04 weight % to about 0.06 weight %, and the composition includes poloxamer 188 in an amount less than about 2.0 weight %, or less than about 1.5 weight %, or less than about 1.0 weight %, or from about 0.4 weight % to about 0.8 weight %, or from about 0.4 weight percent to about 0.6 weight %, based on the total weight of the composition.

In certain embodiments, the presently disclosed compositions may contain water in addition to PHMB and one or more poloxamer compounds. In some embodiments, the presently disclosed compositions are aqueous solutions. For example, the presently disclosed compositions may contain greater than 50 weight % water, based on the total weight of the composition. In certain embodiments, the presently disclosed compositions may include at least 94 weight percent water. In other embodiments, the presently disclosed compositions may include at least 95 weight % water or at least 96 weight % water. In some embodiments, the presently disclosed compositions may include from about 94 weight percent to about 97 weight percent water, or from about 94 weight percent to about 98 weight percent water, or from about 94 weight percent to about 99 weight percent water, based on the total weight of the composition.

In certain embodiments, the presently disclosed solutions may be a saline solution. In such embodiments, the presently disclosed compositions may include sodium chloride (NaCl). For example, the presently disclosed compositions may include from about 0.7 weight percent to about 1.5 weight percent sodium chloride (NaCl), based on the total weight of the composition. In certain embodiments, the presently disclosed compositions may include from about 0.8 weight percent to about 1.1 weight percent sodium chloride (NaCl), based on the total weight of the composition. In still other embodiments, the presently disclosed compositions may include from about 0.85 weight percent to about 0.95 weight percent sodium chloride (NaCl), based on the total weight of the composition. In some embodiments, the presently disclosed compositions may include about 0.90 weight percent sodium chloride (NaCl), based on the total weight of the composition.

According to certain embodiments, the presently disclosed compositions may include PHMB, poloxamer 188, and water. In other embodiments, the presently disclosed compositions may include PHMB, poloxamer 188, BZK, and water. In still other embodiments, the presently disclosed compositions may include PHMB, poloxamer 188, poloxamer 407, and water. In yet other embodiments, the presently disclosed compositions may include PHMB, poloxamer 188, poloxamer 407, BZK, and water. Additionally, the presently disclosed compositions may optionally include sodium chloride (NaCl).

In certain embodiments, the presently disclosed compositions may include from about 0.05 to about 0.15 weight percent PHMB, from about 0.5 to about 5.0 weight percent poloxamer 188, and water. In other embodiments, the presently disclosed compositions may include from about 0.05 to about 0.15 weight percent PHMB, from about 0.5 to about 5.0 weight percent poloxamer 188, from about 0.05 to about 0.15 weight percent BZK, and water. In still other embodiments, the presently disclosed compositions may include from about 0.05 to about 0.15 weight percent PHMB, from about 0.5 to about 2.5 weight percent poloxamer 188, from about 0.5 to about 2.5 weight percent poloxamer 407, and water. In yet other embodiments, the presently disclosed compositions may include from about 0.05 to about 0.15 weight percent PHMB, from about 0.5 to about 2.5 weight percent poloxamer 188, from about 0.5 to about 2.5 weight percent poloxamer 407, from about 0.05 to about 0.15 weight percent BZK, and water.

According to certain embodiments of the present disclosure, the presently disclosed irrigation solution compositions may include from about 0.05 to about 0.1 weight percent PHMB, from about 0.5 to about 2.0 weight percent poloxamer 188, and water. In other embodiments, the presently disclosed irrigation solution compositions may include from about 0.05 to about 0.1 weight percent PHMB, from about 0.5 to about 2.0 weight percent poloxamer 188, from about 0.05 to about 0.13 weight percent BZK, and water. In still other embodiments, the presently disclosed irrigation solution compositions may include from about 0.05 to about 0.1 weight percent PHMB, from about 0.5 to about 2.0 weight percent poloxamer 188, from about 0.5 to about 2.0 weight percent poloxamer 407, and water. In yet other embodiments, the presently disclosed irrigation solution compositions may include from about 0.05 to about 0.1 weight percent PHMB, from about 0.5 to about 2.0 weight percent poloxamer 188, from about 0.5 to about 2.0 weight percent poloxamer 407, from about 0.05 to about 0.13 weight percent BZK, and water.

According to other aspects of the present disclosure, the presently disclosed irrigation solution compositions may include about 0.1 weight percent PHMB, from about 2.0 to about 4.0 weight percent poloxamer 188, and water. In other embodiments, the presently disclosed irrigation solution compositions may include about 0.1 weight percent PHMB, from about 2.0 to about 4.0 weight percent poloxamer 188, about 0.13 weight percent BZK, and water. In still other embodiments, the presently disclosed irrigation solution compositions may include about 0.1 weight percent PHMB, about 2.0 weight percent poloxamer 188, about 2.0 weight percent poloxamer 407, and water. In yet other embodiments, the presently disclosed irrigation solution compositions may include about 0.1 weight percent PHMB, about 2.0 weight percent poloxamer 188, about 2.0 weight percent poloxamer 407, about 0.13 weight percent BZK, and water. In certain embodiments, the presently disclosed irrigation solution compositions may include about 0.1 weight percent PHMB, about 1.0 weight percent poloxamer 188, about 1.0 weight percent poloxamer 407, and water. In some embodiments, the presently disclosed irrigation solution compositions may include about 0.1 weight percent PHMB, about 1.0 weight percent poloxamer 188, about 1.0 weight percent poloxamer 407, about 0.13 weight percent BZK, and water.

According to other aspects of the present disclosure, the presently disclosed irrigation solution compositions may include about 0.05 weight percent PHMB, from about 2.0 to about 4.0 weight percent poloxamer 188, and water. In other embodiments, the presently disclosed irrigation solution compositions may include about 0.05 weight percent PHMB, from about 2.0 to about 4.0 weight percent poloxamer 188, about 0.05 weight percent BZK, and water. In still other embodiments, the presently disclosed irrigation solution compositions may include about 0.05 weight percent PHMB, about 2.0 weight percent poloxamer 188, about 2.0 weight percent poloxamer 407, and water. In yet other embodiments, the presently disclosed irrigation solution compositions may include about 0.05 weight percent PHMB, about 2.0 weight percent poloxamer 188, about 2.0 weight percent poloxamer 407, about 0.05 weight percent BZK, and water. In certain embodiments, the presently disclosed irrigation solution compositions may include about 0.05 weight percent PHMB, about 1.0 weight percent poloxamer 188, about 1.0 weight percent poloxamer 407, and water. In some embodiments, the presently disclosed irrigation solution compositions may include about 0.05 weight percent PHMB, about 1.0 weight percent poloxamer 188, about 1.0 weight percent poloxamer 407, about 0.05 weight percent BZK, and water.

According to another aspect of the present disclosure, the presently disclosed irrigation solution compositions may include from about 0.05 to about 0.15 weight percent PHMB, from about 0.5 to about 2.5 weight percent poloxamer 188, from about 0.5 to about 2.5 weight percent of one or more additional poloxamer compounds, and water. In other embodiments, the presently disclosed irrigation solution compositions may include from about 0.05 to about 0.15 weight percent PHMB, from about 0.5 to about 2.5 weight percent poloxamer 188, from about 0.5 to about 2.5 weight percent of one or more additional poloxamer compounds, from about 0.05 to about 0.15 weight percent BZK, and water. In such embodiments, the one or more additional poloxamer compounds may be selected from the any of the poloxamer compounds presented in Table 1, or may be selected from any poloxamer compounds characterized by an ethylene oxide (EO) content of at least 70%. In still other embodiments, the one or more additional poloxamer compounds may be selected from the group consisting of poloxamer 407, poloxamer 108, poloxamer 1107, poloxamer 338, poloxamer 101, poloxamer 105, poloxamer 122, poloxamer 123, poloxamer 124, poloxamer 181, poloxamer 182, poloxamer 183, poloxamer 184, poloxamer 185, poloxamer 212, poloxamer 215, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 401, poloxamer 402, poloxamer 403, poloxamer 105 benzoate, poloxamer 182 dibenzoate, and any combination thereof. In other embodiments, the one or more additional poloxamer compounds may be selected from the group consisting of poloxamer 407, poloxamer 108, poloxamer 1107, poloxamer 338, and any combination thereof. In still other embodiments, the one or more additional poloxamer compounds may be selected from the group consisting of poloxamer 407, poloxamer 108, poloxamer 338, poloxamer 217, poloxamer 237, poloxamer 238, poloxamer 288, and any combination thereof. In other embodiments, the one or more additional poloxamer compounds may be selected from the group consisting of poloxamer 407, poloxamer 108, poloxamer 338, poloxamer 217, poloxamer 237, poloxamer 238, poloxamer 288, poloxamer 1107, and any combination thereof. In certain embodiments, the one or more additional poloxamer compounds may include a mixture of poloxamer 407 and poloxamer 1107, or a mixture of poloxamer 108 and poloxamer 407, or a mixture of poloxamer 108 and poloxamer 338, or a mixture of poloxamer 217, poloxamer 237, and poloxamer 238.

Any of the presently disclosed irrigation solution compositions may optionally include sodium chloride (NaCl) or otherwise be a saline solution suitable for irrigation of the implant pocket in a surgical setting. For example, the presently disclosed irrigation solution compositions may include from about 0.7 weight percent to about 1.5 weight percent NaCl, or more preferably, from about 0.8 weight percent to about 1.1 weight percent NaCl, based on the total weight of the composition. In general, the remainder of the presently disclosed irrigation solution compositions is water. For example, the presently disclosed irrigation solution compositions may include from about 94 weight percent to about 99 weight percent water, or from about 93.7 weight percent to about 98 weight percent water, based on the total weight of the composition.

In certain embodiments, the presently disclosed irrigation solution compositions do not include betaine, trimethylglycine (TMG), or povidone-iodine or specifically exclude betaine, trimethylglycine (TMG), and povidone-iodine. In such embodiments, the presently disclosed irrigation solution compositions are effective at improving one or more patient outcomes, including enhanced removal of silicone gel from the implant pocket, decreased post-surgical infection rates, decreased capsular contracture rates, and decreased complications resulting from surgical implant insertion and/or removal.

The presently disclosed irrigation solution compositions are particularly well-suited for the irrigation and treatment of an implant pocket prior to insertion or following the removal of a silicone implant. More particularly, the presently disclosed irrigation solution compositions are operable to decrease the risk of infection and capsular contracture resulting from implant insertion and removal, as well as mitigate or reduce the complications coincident with surgical insertion or removal of prosthetic implants.

According to one aspect of the present disclosure, a method of irrigating a surgically-created implant pocket in a subject is provided. The method may include contacting the implant pocket with any of the presently disclosed compositions and irrigation solutions. The term “implant pocket,” referred to in the presently disclosed methods, refers to a surgically-created pocket in a subject that is intended to contain a prosthetic implant or that is created by a surgeon for the purpose of facilitating the insertion of a prosthetic implant.

In at least certain embodiments, contacting the implant pocket with the composition or irrigation solution may include causing an irrigation solution to pass through a dissection tunnel connecting the implant pocket to an incision on the skin of a subject such that it enters and at least partially fills the implant pocket. In other embodiments, contacting the implant pocket with the composition or irrigation solution may include rinsing the implant pocket with the composition or irrigation solution.

Contacting the implant pocket with an irrigation solution composition, according to the presently disclosed methods and compositions, may occur or be performed either before a prosthetic implant is inserted into the implant pocket or after a prosthetic implant is removed from the implant pocket. For example, contacting the implant pocket with an irrigation solution composition may occur or be performed shortly following the surgical creation of the implant pocket and before any prosthetic implant has been inserted into the implant pocket. In such instances, contacting the implant pocket with an irrigation solution composition may occur or be performed following the surgical creation of the implant pocket and immediately prior to insertion of the prosthetic implant into the implant pocket. In other cases, contacting the implant pocket with an irrigation solution composition may occur or be performed following the removal of a prosthetic implant from the implant pocket. In such cases, an implant that was previously inserted into the implant pocket during an earlier surgery may be removed for a variety of potential reasons, including, but not limited to, failure of the prosthetic implant, undesirable implant functioning or aesthetics, a desire by the subject that the prosthetic implant be removed or replaced, a need to replace the prosthetic implant with a newer or different prosthetic implant, to correct the placement or functioning of the prosthetic implant, and to remove a ruptured or faulty prosthetic implant.

In cases in which the contacting the implant pocket with an irrigation solution composition occurs or is performed following the removal of a prosthetic implant from the implant pocket, a capsulectomy may optionally be performed on the implant pocket following implant removal and prior to contacting the implant pocket with the irrigation solution. A capsulectomy is a surgical procedure that removes scar tissue and/or at least a portion of the capsule tissue that formed around the prosthetic implant. The capsulectomy may be a partial capsulectomy, a total capsulectomy, or an en bloc capsulectomy. In at least some embodiments, the implant pocket that is contacted with the irrigation solution composition may be at least partially formed as a result of a capsulectomy.

In certain embodiments, the method of irrigating a surgically-created implant pocket may further include removing at least a portion of the irrigation solution or substantially removing the irrigation solution from the implant pocket. In certain embodiments, removing the irrigation solution, or at least a portion thereof, may include causing, via suction, the irrigation solution, or at least a portion thereof, to exit the implant pocket and pass through the dissection tunnel so as to be removed from the subject. In certain embodiments, the method may further include repeating both the contacting the implant pocket with the irrigation solution and the removing the irrigation solution from the implant pocket steps two (2) to six (6) times. The method of irrigating a surgically-created implant pocket may further include rinsing the implant pocket with a sterile saline solution. In at least certain embodiments, rinsing the implant pocket with a sterile saline solution may include causing a sterile saline solution to pass through a dissection tunnel connecting the implant pocket to an incision on a skin of a subject such that it enters and at least partially fills the implant pocket. Rinsing the implant pocket with a sterile saline solution may also include, in certain embodiments, causing, via suction, the sterile saline solution, or at least a portion thereof, to exit the implant pocket and pass through the dissection tunnel so as to be removed from the subject. According to certain embodiments, the method of irrigating a surgically-created implant pocket may further include inserting a prosthetic implant into the surgically-created implant pocket.

The prosthetic implant that may be inserted into the surgically-created implant pocket or that may be intended to be inserted into an implant pocket, according to the presently disclosed methods, may include filled implants or pre-filled implants, unfilled implants, saline implants, silicone gel implants, textured implants, smooth implants, highly cohesive silicone gel implants, oil-filled implants. In other embodiments, the prosthetic implant may be selected from the group consisting of breast implants, gluteal implants, buttock implants, and facial implants. In other embodiments, the prosthetic implant may be a silicone implant or a silicone gel implant selected from the group consisting of silicone breast implants, silicone gluteal implants, silicone buttock implants, and silicone facial implants. In still other embodiments, the prosthetic implant may be selected from the group consisting of an implantable device, a pacemaker, a joint replacement prosthesis, a tissue graft, an allograft, and an autograft.

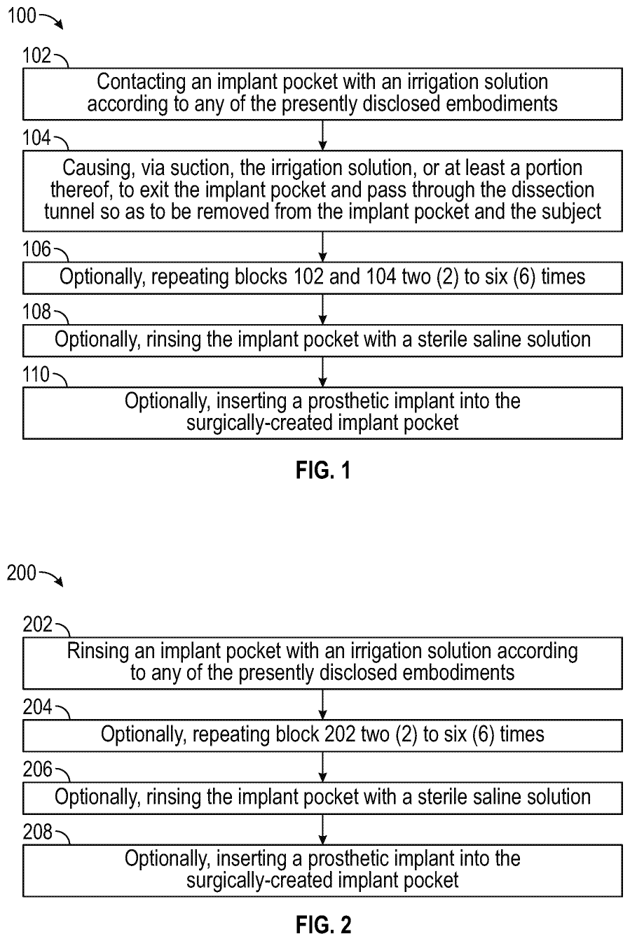

FIG. 1 provides a diagrammatic representation of an example method for irrigation of a surgically-created implant pocket in a subject, according to an exemplary embodiment of the present disclosure. As shown in FIG. 1, method 100 for irrigating a surgically-created implant pocket may include, at block 102, contacting an implant pocket with an irrigation solution according to any one of the presently disclosed irrigation solution compositions. At block 104, method 100 may further include causing, via suction, the irrigation solution, or at least a portion thereof, to exit the implant pocket and pass through the dissection tunnel so as to be removed from the implant pocket and the subject. At block 106, method 100 may optionally include repeating blocks 102 and 104 two (2) to six (6) times. Method 100 may further optionally include, at block 108, rinsing the implant pocket with a sterile saline solution. At block 110, method 100 may also optionally include inserting a prosthetic implant into the surgically-created implant pocket.

FIG. 2 provides a diagrammatic representation of an example alternative method for irrigating a surgically-created implant pocket in a subject, according to an exemplary embodiment of the present disclosure. Method 200 for irrigating a surgically-created implant pocket in a subject may include, at block 202, rinsing an implant pocket with an irrigation solution according to any of the presently disclosed embodiments. At block 204, method 200 may further optionally include repeating block 202 two (2) to six (6) times. Method 200 may further optionally include, at block 206, rinsing the implant pocket with a sterile solution. At block 208, method 200 may also optionally include inserting a prosthetic implant into the surgically-created implant pocket.

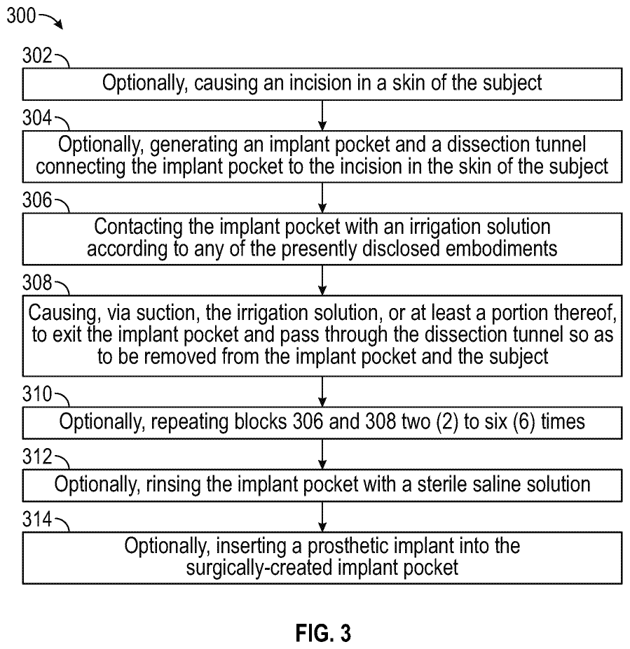

FIG. 3 provides a diagrammatic representation of another example method for irrigating an implant pocket in a subject during surgical insertion of a prosthetic implant into a subject, according to an exemplary embodiment of the present disclosure. Method 300 for irrigating an implant pocket in a subject may optionally include, at block 302, causing an incision in a skin of the subject. At block 304, method 300 may optionally include generating an implant pocket and a dissection tunnel connecting the implant pocket to the incision in the skin of the subject. Method 300 may include, at block 306, contacting the implant pocket with any of the presently disclosed irrigation solutions or compositions. At block 308, method 300 may also include causing, via suction, the irrigation solution, or at least a portion thereof, to exit the implant pocket and pass through the dissection tunnel so as to be removed from the implant pocket and the subject. Method 300 may optionally include, at block 310, repeating blocks 306 and 308 two (2) to six (6) times. At block 312, method 300 may also optionally include rinsing the implant pocket with a sterile saline solution. Method 314 may also optionally include, at block 314, inserting a prosthetic implant into the surgically-created implant pocket.

Method 100, method 200, or method 300 may be performed on a fresh surgically-created implant pocket before an implant has been inserted or may be performed on an implant pocket after a previously inserted implant has been removed. According to at least certain aspects of the present disclosure, the implant pocket in any one of methods 100, 200, and 300 may be a breast implant pocket and the prosthetic implant in any one of methods 100, 200, and 300 may be a breast implant. In instances in which the implant pocket in method 300 is a breast implant pocket, the optional causing an incision in the skin of the subject may include an incision selected from the group consisting of an inframammary incision, a periareolar incision, an axillary incision, and a vertical incision (part of mastopexy pattern).

In certain embodiments, the method of irrigating a surgically-created implant pocket in a subject may include: (a) optionally, causing an incision in a skin of the subject; (b) optionally, generating an implant pocket and a dissection tunnel connecting the implant pocket to the incision in the skin of the subject; (c) contacting the implant pocket with any of the presently disclosed irrigation solutions or compositions; (d) causing, via suction, the irrigation solution, or at least a portion thereof, to exit the implant pocket and pass through the dissection tunnel so as to be removed from the implant pocket and the subject; (e) optionally, repeating steps (c)-(d) two (2) to six (6) times; (f) optionally, rinsing the implant pocket with a sterile saline solution; (g) optionally, inserting a prosthetic implant into the surgically-created implant pocket. In at least certain embodiments, contacting the implant pocket with the irrigation solution may include causing an irrigation solution to pass through a dissection tunnel connecting the implant pocket to an incision on the skin of a subject such that it enters and at least partially fills the implant pocket. In other embodiments, contacting the implant pocket with the irrigation solution may include rinsing the implant pocket with the composition or irrigation solution. The method may also include causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.

The applicant has unexpectedly discovered that the presently disclosed irrigation solutions and compositions may be particularly effective to facilitate removal of the silicone gel from an implant pocket resulting from the rupture of a silicone gel implant, thereby improving patient outcomes for patients suffering from a ruptured silicone gel implant. In particular, the presently disclosed compositions and methods may be operable to reduce the risk of granuloma formation, inflammation, infection, and systemic complications resulting from silicone gel leakage following the removal of a ruptured silicone gel implant from an implant pocket.

Accordingly, the present disclosure provides methods of removing silicone gel or silicone residue from a surgically-created implant pocket in a subject. In exemplary embodiments, the method may include contacting an implant pocket containing silicone gel, or silicone residue that originated from a silicone gel implant, with any of the presently disclosed irrigation solutions or compositions. In at least certain embodiments, the silicone gel or silicone residue in the implant pocket may be derived from or be the result of a ruptured silicone gel implant. In at least certain embodiments, contacting the implant pocket with the irrigation solution may include causing an irrigation solution to pass through a dissection tunnel connecting the implant pocket to an incision on the skin of a subject such that it enters and at least partially fills the implant pocket. In other embodiments, contacting the implant pocket with the irrigation solution may include rinsing the implant pocket with the composition or irrigation solution. The method may also include removing at least a portion of the irrigation solution from the implant pocket or substantially removing the irrigation solution from the implant pocket. The method may also include causing, via suction, the irrigation solution, or at least a portion thereof, to exit the implant pocket and pass through the dissection tunnel so as to be removed from the subject. The method may also include causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject. In certain embodiments, the implant pocket may be a breast implant pocket and the ruptured silicone gel implant may be a ruptured silicone gel breast implant. In other embodiments, implant pocket may be a gluteal implant pocket and the ruptured silicone gel implant may be a ruptured silicone gel gluteal implant.

According to another aspect of the present disclosure, a method of treating a subject having a surgically-created implant pocket that contains silicone gel or that has been exposed to silicone residue as a result of the rupture of a silicone gel implant. In exemplary embodiments, the method may include contacting an implant pocket containing silicone gel or silicone residue that is derived from a ruptured silicone gel implant with any of the presently disclosed irrigation solutions or compositions. In at least certain embodiments, contacting the implant pocket with the irrigation solution may include causing an irrigation solution to pass through a dissection tunnel connecting the implant pocket to an incision on the skin of a subject such that it enters and at least partially fills the implant pocket. In other embodiments, contacting the implant pocket with the irrigation solution may include rinsing the implant pocket with the composition or irrigation solution. The method may also include removing at least a portion of the irrigation solution from the implant pocket or substantially removing the irrigation solution from the implant pocket. The method may also include causing, via suction, the irrigation solution, or at least a portion thereof, to exit the implant pocket and pass through the dissection tunnel so as to be removed from the subject. The method may also include causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject. In certain embodiments, the implant pocket may be a breast implant pocket and the ruptured silicone gel implant may be a ruptured silicone gel breast implant. In other embodiments, implant pocket may be a gluteal implant pocket and the ruptured silicone gel implant may be a ruptured silicone gel gluteal implant.

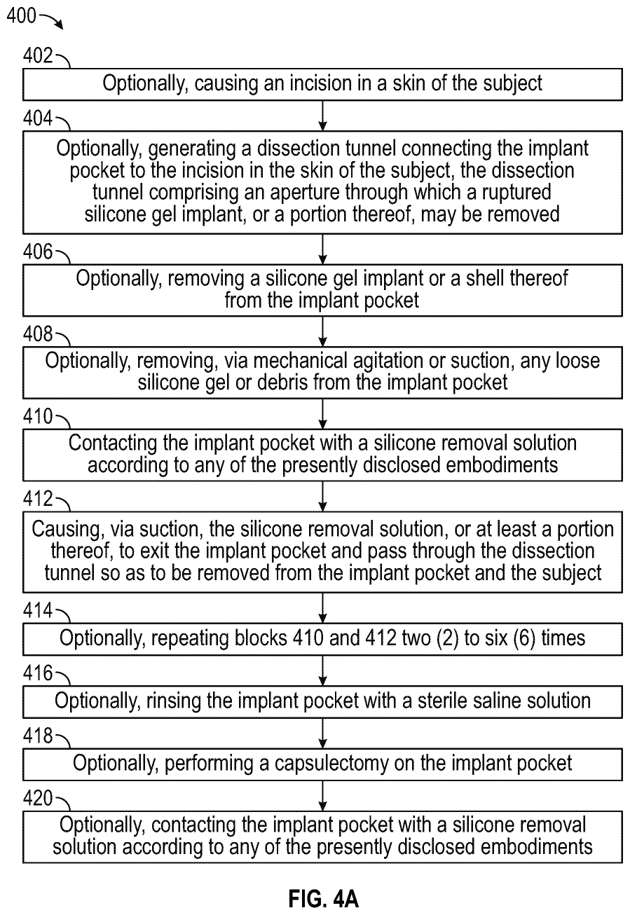

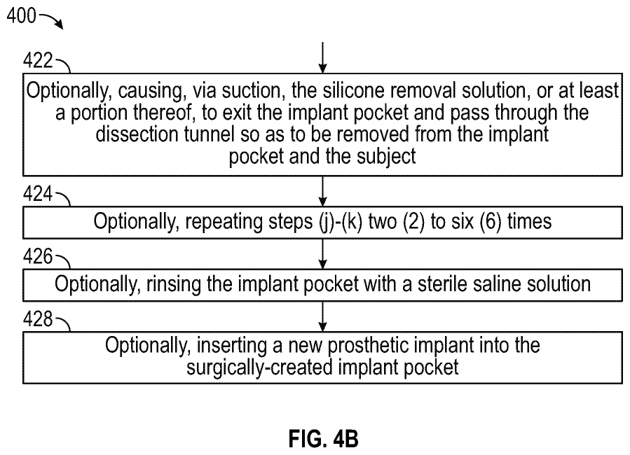

FIGS. 4A-4B provides a diagrammatic representation of an example method for treating an implant pocket or removing silicone from an implant pocket during the removal or replacement of a silicone gel implant in a subject, according to an exemplary embodiment of the present disclosure. Method 400 for treating an implant pocket or removing silicone from an implant pocket during the removal or replacement of a silicone gel implant in a subject may optionally include, at block 402, causing an incision in a skin of the subject. At block 404, method 400 may optionally include generating a dissection tunnel connecting the implant pocket to the incision in the skin of the subject. For example, the dissection tunnel may include an aperture through which a ruptured silicone gel implant, or a portion thereof, may be removed from the implant pocket.

Method 400 may also optionally include, at block 406, removing a silicone gel implant or a shell thereof from the implant pocket. At block 408, method 400 may also optionally include removing, via mechanical agitation or suction, any loose silicone gel or debris from the implant pocket. Method 400 may also include, at block 410, contacting the implant pocket with a silicone removal solution (e.g., an irrigation solution), according to any of the presently disclosed irrigation solutions and compositions. At block 412, method 400 may also include causing, via suction, the silicone removal solution, or at least a portion thereof, to exit the implant pocket and pass through the dissection tunnel so as to be removed from the implant pocket and the subject. Optionally, method 400 may also include, at block 414, repeating block 410 and 412 two (2) to six (6) times. At block 416, method 400 may also optionally include rinsing the implant pocket with a sterile saline solution. Method 400 may also optionally include, at bock 418, performing a capsulectomy on the implant pocket. At block 420, method 400 may also optionally include contacting the implant pocket with a silicone removal solution (e.g., an irrigation solution), according to any of the presently disclosed irrigation solutions and compositions.

Method 400 may also optionally include, at block 422, causing, via suction, the silicone removal solution, or at least a portion thereof, to exit the implant pocket and pass through the dissection tunnel so as to be removed from the implant pocket and the subject. At block 424, method 400 may also optionally include repeating blocks 420 and 422 two (2) to six (6) times. Method 400 may also optionally include, at block 426, rinsing the implant pocket with a sterile saline solution. At block 428, method 400 may also optionally include inserting a new prosthetic implant into the surgically-created implant pocket.

In certain embodiments, method 400 may be a method of treating a breast implant pocket or removing silicone from a breast implant pocket during the removal or replacement of a silicone gel breast implant in a subject. In such instances, the implant pocket in method 400 may be a breast implant pocket and the ruptured silicone gel implant may be a ruptured silicone gel breast implant. In instances in which the implant pocket in method 400 is a breast implant pocket, the optional causing an incision in the skin of the subject may include an incision selected from the group consisting of an inframammary incision, a periareolar incision, an axillary incision, and a vertical incision (part of mastopexy pattern).

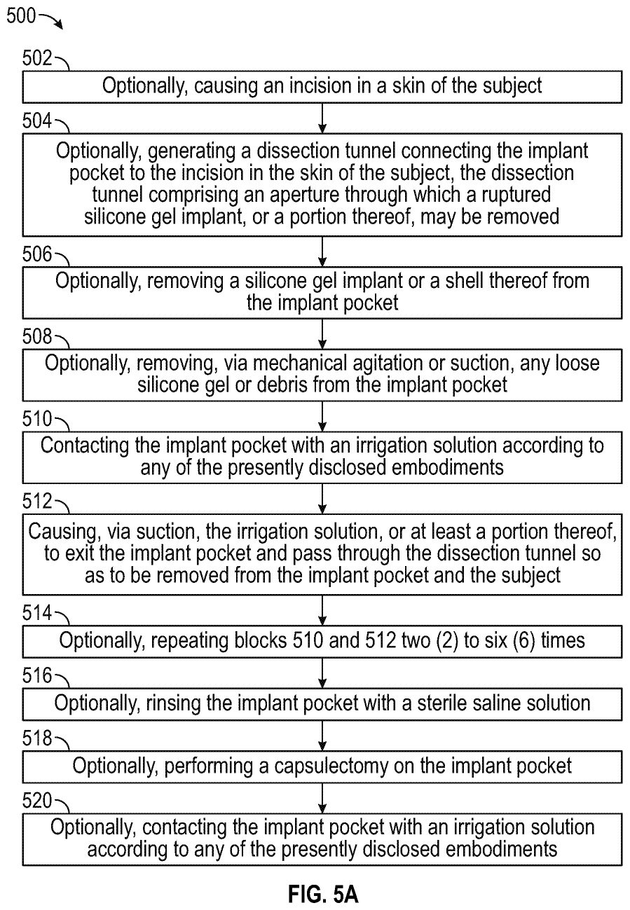

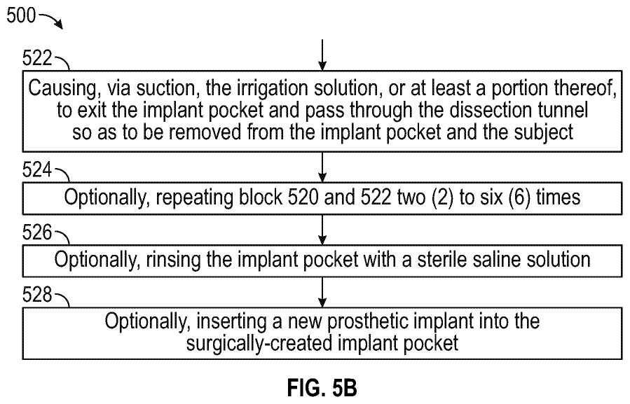

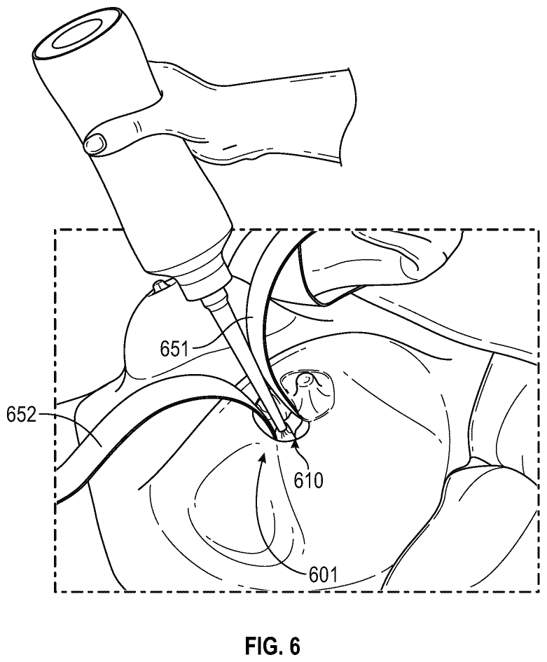

FIGS. 5A-5B provides a diagrammatic representation of an example method of removing a ruptured silicone implant from an implant pocket in a subject or replacing a ruptured silicone gel implant in a subject in need thereof, according to an exemplary embodiment of the present disclosure. Method 500 for removing or replacing a ruptured silicone gel implant in a subject in need thereof may optionally include, at block 502, causing an incision in a skin of the subject. At block 504, method 500 may optionally include generating a dissection tunnel connecting the implant pocket containing the ruptured silicone gel implant to the incision in the skin of the subject. For example, the dissection tunnel may include an aperture through which a ruptured silicone gel implant, or a portion thereof, may be removed from the implant pocket.

Method 500 may also optionally include, at block 506, removing a silicone gel implant or a shell thereof from the implant pocket. At block 508, method 500 may also optionally include removing, via mechanical agitation or suction, any loose silicone gel or debris from the implant pocket. Method 500 may also include, at block 510, contacting the implant pocket with an irrigation solution according to any of the presently disclosed irrigation solutions and compositions. At block 512, method 500 may also include causing, via suction, the silicone removal solution, or at least a portion thereof, to exit the implant pocket and pass through the dissection tunnel so as to be removed from the implant pocket and the subject. Optionally, method 500 may also include, at block 514, repeating block 510 and 512 two (2) to six (6) times. At block 516, method 500 may also optionally include rinsing the implant pocket with a sterile saline solution. Method 500 may also optionally include, at bock 518, performing a capsulectomy on the implant pocket. At block 520, method 500 may also optionally include contacting the implant pocket with an irrigation solution according to any of the presently disclosed irrigation solutions and compositions.

Method 500 may also optionally include, at block 522, causing, via suction, the silicone removal solution, or at least a portion thereof, to exit the implant pocket and pass through the dissection tunnel so as to be removed from the implant pocket and the subject. At block 524, method 500 may also optionally include repeating blocks 520 and 522 two (2) to six (6) times. Method 500 may also optionally include, at block 526, rinsing the implant pocket with a sterile saline solution. At block 528, method 500 may also optionally include inserting a new prosthetic implant into the surgically-created implant pocket.

In certain embodiments, method 500 may be a method of removing or replacing a ruptured silicone gel breast implant in a subject in need thereof. In such instances, the implant pocket in method 500 may be a breast implant pocket and the ruptured silicone gel implant may be a ruptured silicone gel breast implant. In instances in which the implant pocket in method 500 is a breast implant pocket, the optional causing an incision in the skin of the subject may include an incision selected from the group consisting of an inframammary incision, a periareolar incision, an axillary incision, and a vertical incision (part of mastopexy pattern).

According to another aspect of the present disclosure, a method of removing silicone gel or silicone residue from a surgically-created implant pocket in a subject is provided. The method may include: (a) optionally, causing an incision in a skin of the subject; (b) optionally, generating a dissection tunnel connecting the implant pocket to the incision in the skin of the subject, the dissection tunnel comprising an aperture through which a ruptured silicone gel implant, or a portion thereof, may be removed; (c) optionally, removing a silicone gel implant or a shell thereof from the implant pocket; (d) optionally, removing, via mechanical agitation or suction, any loose silicone gel or debris from the implant pocket; (e) contacting the implant pocket with any of the presently disclosed silicone removal solutions or compositions; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with any of the presently disclosed silicone removal solutions or compositions; (k) optionally, causing, via suction, the silicone removal solution, or at least a portion thereof, to exit the implant pocket and pass through the dissection tunnel so as to be removed from the implant pocket and the subject; (l) optionally, repeating steps (j)-(k) two (2) to six (6) times; (m) optionally, rinsing the implant pocket with a sterile saline solution; and (n) optionally, inserting a new prosthetic implant into the surgically-created implant pocket. In certain embodiments, the new prosthetic implant may be selected from the group consisting of filled implants or pre-filled implants, unfilled implants, saline implants, silicone gel implants, textured implants, smooth implants, highly cohesive silicone gel implants, and oil-filled implants. In other embodiments, the new prosthetic implant may be selected from the group consisting of breast implants, gluteal implants, buttock implants, and facial implants. In certain embodiments, the silicone gel implant or ruptured silicone gel implant may be selected from the group consisting of silicone gel breast implants, silicone gel gluteal implants, silicone gel buttock implants, and silicone gel facial implants.

According to at least one aspect of the present disclosure, a method of treating a subject in need thereof and having a surgically-created implant pocket that contains silicone gel or that has been exposed to silicone as a result of the rupture of a silicone gel implant is provided. The method may include: (a) optionally, causing an incision in a skin of the subject; (b) optionally, generating a dissection tunnel connecting the implant pocket to the incision in the skin of the subject, the dissection tunnel comprising an aperture through which a ruptured silicone gel implant may be removed; (c) optionally, removing a silicone gel implant or a shell thereof from the implant pocket; (d) optionally, removing, via mechanical agitation or suction, any loose silicone gel or debris from the implant pocket; (e) contacting the implant pocket with a silicone removal solution according to any of the presently disclosed compositions; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with a silicone removal solution according to any of the presently disclosed compositions; (k) optionally, causing, via suction, the silicone removal solution, or at least a portion thereof, to exit the implant pocket and pass through the dissection tunnel so as to be removed from the implant pocket and the subject; (l) optionally, repeating steps (j)-(k) two (2) to six (6) times; (m) optionally, rinsing the implant pocket with a sterile saline solution; and (n) optionally, inserting a new prosthetic implant into the surgically-created implant pocket. In certain embodiments, the new prosthetic implant may be selected from the group consisting of filled implants or pre-filled implants, unfilled implants, saline implants, silicone gel implants, textured implants, smooth implants, highly cohesive silicone gel implants, and oil-filled implants. In other embodiments, the new prosthetic implant may be selected from the group consisting of breast implants, gluteal implants, buttock implants, and facial implants. In certain embodiments, the silicone gel implant or ruptured silicone gel implant may be selected from the group consisting of silicone gel breast implants, silicone gel gluteal implants, silicone gel buttock implants, and silicone gel facial implants.

According to another aspect of the present disclosure, a method of removing a ruptured silicone gel implant from a subject in need thereof is provided. The method may include: (a) optionally, causing an incision in a skin of the subject; (b) optionally, generating a dissection tunnel connecting an implant pocket in the subject to the incision in the skin of the subject, wherein the implant pocket contains a ruptured silicone gel implant and the dissection tunnel comprises an aperture through which a ruptured silicone gel implant may be removed; (c) optionally, removing the ruptured silicone gel implant or a shell thereof from the implant pocket; (d) optionally, removing, via mechanical agitation or suction, any loose silicone gel or debris from the implant pocket; (e) contacting the implant pocket with a silicone removal solution according to any of the presently disclosed compositions; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with a silicone removal solution according to any of the presently disclosed compositions; (k) optionally, causing, via suction, the silicone removal solution, or at least a portion thereof, to exit the implant pocket and pass through the dissection tunnel so as to be removed from the implant pocket and the subject; (l) optionally, repeating steps (j)-(k) two (2) to six (6) times; (m) optionally, rinsing the implant pocket with a sterile saline solution; and (n) optionally, inserting a new prosthetic implant into the surgically-created implant pocket. In certain embodiments, the new prosthetic implant may be selected from the group consisting of filled implants or pre-filled implants, unfilled implants, saline implants, silicone gel implants, textured implants, smooth implants, highly cohesive silicone gel implants, and oil-filled implants. In other embodiments, the new prosthetic implant may be selected from the group consisting of breast implants, gluteal implants, buttock implants, and facial implants. In certain embodiments, the silicone gel implant or ruptured silicone gel implant may be selected from the group consisting of silicone gel breast implants, silicone gel gluteal implants, silicone gel buttock implants, and silicone gel facial implants.

According to another aspect of the present disclosure, a method of replacing a silicone gel implant in a subject in need thereof, the subject having a surgically-created implant pocket that contains a silicone gel implant. The method may include: (a) optionally, causing an incision in a skin of the subject; (b) optionally, generating a dissection tunnel connecting the implant pocket to the incision in the skin of the subject, the dissection tunnel comprising an aperture through which the silicone gel implant may be removed; (c) optionally, removing the silicone gel implant or a shell thereof from the implant pocket; (d) optionally, removing, via mechanical agitation or suction, any loose silicone gel or debris from the implant pocket; (e) contacting the implant pocket with an irrigation solution according to any one of the presently disclosed compositions; (f) causing, via suction, the irrigation solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with an irrigation solution according to any of the presently disclosed compositions; (k) optionally, causing, via suction, the irrigation solution, or at least a portion thereof, to exit the implant pocket and pass through the dissection tunnel so as to be removed from the implant pocket and the subject; (l) optionally, repeating steps (c)-(d) two (2) to six (6) times; (m) optionally, rinsing the implant pocket with a sterile saline solution; and (n) optionally, inserting a new prosthetic implant into the surgically-created implant pocket. In certain embodiments, the silicone gel implant in the implant pocket may be intact. In other embodiments, the silicone gel implant in the implant pocket of the subject is ruptured. In certain embodiments, the implant pocket contains silicone gel derived from a ruptured silicone gel implant. In certain embodiments, the new prosthetic implant may be selected from the group consisting of filled implants or pre-filled implants, unfilled implants, saline implants, silicone gel implants, textured implants, smooth implants, highly cohesive silicone gel implants, and oil-filled implants. In other embodiments, the new prosthetic implant may be selected from the group consisting of breast implants, gluteal implants, buttock implants, and facial implants. In certain embodiments, the silicone gel implant may be selected from the group consisting of silicone gel breast implants, silicone gel gluteal implants, silicone gel buttock implants, and silicone gel facial implants.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

### EXAMPLES

### Examples 1-44—Exemplary Irrigation Solution Compositions

Table 2 provides non-exhaustive and non-limiting exemplary irrigation solution compositions, according to exemplary embodiments of the present disclosure. In addition to the compositional components disclosed in Table 2, Examples 1-44 may further include other compositional components, including water and, optionally, sodium chloride. In certain embodiments, the compositions provided in Examples 1-44 may optionally include from about 0.7 weight percent to about 1.5 weight percent NaCl, or more preferably, from about 0.8 weight percent to about 1.1 weight percent NaCl, based on the total weight of the composition. In general, the remainder of the presently disclosed irrigation solution compositions may be water. For example, Examples 1-44 may include from about 94 weight percent to about 99 weight percent water, or from about 93.7 weight percent to about 98 weight percent water, based on the total weight of the composition.

TABLE 2

| | | Poloxamer | Poloxamer | Other | |
|---|---|---|---|---|---|
| | PHMB | 188 | 407 | Poloxamer | BZK |
| Example | (wt %) | (wt %) | (wt %) | (wt %) | (wt %) |
| 1 | 0.05-0.15 | 0.5-5.0 | — | — | — |
| 2 | 0.05-0.15 | 0.5-5.0 | — | — | 0.05-0.15 |
| 3 | 0.05-0.15 | 0.5-2.5 | 0.5-2.5 | — | — |
| 4 | 0.05-0.15 | 0.5-2.5 | 0.5-2.5 | — | 0.05-0.15 |
| 5 | 0.05-0.1 | 0.5-2.0 | — | — | — |
| 6 | 0.05-0.1 | 0.5-2.0 | — | — | 0.05-0.13 |
| 7 | 0.05-0.1 | 0.5-2.0 | 0.5-2.0 | — | — |
| 8 | 0.05-0.1 | 0.5-2.0 | 0.5-2.0 | — | 0.05-0.13 |
| 9 | 0.1 | 2.0-4.0 | — | — | — |
| 10 | 0.1 | 2.0-4.0 | — | — | 0.13 |
| 11 | 0.1 | 2.0 | 2.0 | — | — |
| 12 | 0.1 | 2.0 | 2.0 | — | 0.13 |
| 13 | 0.1 | 1.0 | 1.0 | — | — |
| 14 | 0.1 | 1.0 | 1.0 | — | 0.13 |
| 15 | 0.05 | 2.0-4.0 | — | — | — |
| 16 | 0.05 | 2.0-4.0 | — | — | 0.05 |
| 17 | 0.05 | 2.0 | 2.0 | — | — |
| 18 | 0.05 | 2.0 | 2.0 | — | 0.05 |
| 19 | 0.05 | 1.0 | 1.0 | — | — |
| 20 | 0.05 | 1.0 | 1.0 | — | 0.05 |
| 21 | 0.05-0.15 | 0.5-2.5 | — | 0.5-2.5 | — |
| 22 | 0.05-0.15 | 0.5-2.5 | — | 0.5-2.5 | 0.05-0.15 |
| 23 | 0.05-0.15 | 0.5-65.0 | — | — | — |
| 24 | 0.05-0.15 | 0.5-65.0 | — | — | 0.05-0.15 |
| 25 | 0.05-0.15 | 12.5 | 12.5 | — | — |
| 26 | 0.05-0.15 | 15.0 | 7.5 | — | 0.05-0.15 |
| 27 | 0.05-0.1 | 25.0-35.0 | — | — | — |
| 28 | 0.05-0.1 | 5.0-15.0 | — | — | 0.05-0.13 |
| 29 | 0.05-0.1 | 10.0-20.0 | 10.0-20.0 | — | — |
| 30 | 0.05-0.1 | 5.0-15.0 | 5.0-15.0 | — | 0.05-0.13 |
| 31 | 0.1 | 5-15 | — | — | — |
| 32 | 0.1 | 0.5-15 | — | — | 0.13 |
| 33 | 0.1 | 7.5 | 7.5 | — | — |
| 34 | 0.1 | 5.0 | 5.0 | — | 0.13 |
| 35 | 0.1 | 2.5 | 2.5 | — | — |
| 36 | 0.1 | 25.0 | 5.0 | — | 0.13 |
| 37 | 0.05 | 0.5-50.0 | — | — | — |
| 38 | 0.05 | 0.5-50.0 | — | — | 0.05 |
| 39 | 0.05 | 25.0 | 5.0 | — | — |
| 40 | 0.05 | 30.0 | 2.5 | — | 0.05 |
| 41 | 0.05 | 15.0 | 15.0 | — | — |
| 42 | 0.05 | 15.0 | 15.0 | — | 0.05 |
| 43 | 0.05-0.15 | 0.5-35.0 | — | 0.5-15.0 | — |
| 44 | 0.05-0.15 | 0.5-35.0 | — | 0.5-15.0 | 0.05-0.15 |

### Example 45—Irrigation Solution Compositions are Effective for the Treatment of an Implant Pocket Prior to Insertion of a Prosthetic Implant

An irrigation solution composition according to any one of Examples 1-44 will be used to treat a surgically-created implant pocket prior to the insertion of a prosthetic implant, according to the presently disclosed methods. Treatment of the implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, and a reduction in one or more post-surgical complications.

### Example 46—Irrigation Solution Compositions are Effective for the Treatment of an Implant Pocket Prior Following the Removal of a Prosthetic Implant

An irrigation solution composition according to any one of Examples 1-44 will be used to treat a surgically-created implant pocket following the removal of a prosthetic implant, according to the presently disclosed methods. Treatment of the implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, and a reduction in one or more post-surgical complications.

### Example 47—Irrigation Solution Compositions are Effective for the Treatment of an Implant Pocket Following the Removal of a Silicone Gel Implant

An irrigation solution composition according to any one of Examples 1-44 will be used to treat a surgically-created implant pocket following the removal of a silicone gel implant, according to the presently disclosed methods. Treatment of the implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, reduced rate or risk of granuloma formation, increased silicone gel removal from the implant pocket, and a reduction in one or more post-surgical complications.

### Example 48—Irrigation Solution Compositions are Effective for the Removal of Silicone Gel from an Implant Pocket in a Subject Following the Removal of a Ruptured Silicone Gel Implant from the Implant Pocket

An irrigation solution composition according to any one of Examples 1-44 will be used to treat a surgically-created implant pocket following the removal of a ruptured silicone gel implant, according to the presently disclosed methods. Treatment of the implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, reduced rate or risk of granuloma formation, increased silicone gel removal from the implant pocket, and a reduction in one or more post-surgical complications.

### Example 49—Irrigation Solution Compositions are Effective for the Treatment of a Breast Implant Pocket Prior to Insertion of a Breast Implant

An irrigation solution composition according to any one of Examples 1-44 will be used to treat a surgically-created breast implant pocket prior to the insertion of a breast implant, according to the presently disclosed methods. Treatment of the breast implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, and a reduction in one or more post-surgical complications.

### Example 50—Irrigation Solution Compositions are Effective for the Treatment of a Breast Implant Pocket Prior Following the Removal of a Breast Implant

An irrigation solution composition according to any one of Examples 1-44 will be used to treat a surgically-created breast implant pocket following the removal of a breast implant, according to the presently disclosed methods. Treatment of the breast implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, and a reduction in one or more post-surgical complications.

### Example 51—Irrigation Solution Compositions are Effective for the Treatment of a Breast Implant Pocket Following the Removal of a Silicone Gel Breast Implant

An irrigation solution composition according to any one of Examples 1-44 will be used to treat a surgically-created breast implant pocket following the removal of a silicone gel breast implant, according to the presently disclosed methods. Treatment of the breast implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, reduced rate or risk of granuloma formation, increased silicone gel removal from the implant pocket, and a reduction in one or more post-surgical complications.

### Example 52—Irrigation Solution Compositions are Effective for the Removal of Silicone Gel from a Breast Implant Pocket in a Subject Following the Removal of a Ruptured Silicone Gel Breast Implant from the Breast Implant Pocket

An irrigation solution composition according to any one of Examples 1-44 will be used to treat a surgically-created breast implant pocket following the removal of a ruptured silicone gel breast implant, according to the presently disclosed methods. Treatment of the breast implant pocket using an irrigation solution according to any one of Examples 1-44 will improve one or more patient outcomes, including, but not limited to, decreased risk of infection, decreases capsular contracture rate, a reduction in post-surgical inflammation, reduced post-surgical scar tissue development, reduced biofilm development around the implant, reduced rate or risk of granuloma formation, increased silicone gel removal from the implant pocket, and a reduction in one or more post-surgical complications.

### STATEMENTS OF THE DISCLOSURE

Statement 1. A composition for irrigating a surgically-created implant pocket in a subject, the composition comprising: polyhexamethylene biguanide (PHMB); one or more poloxamer compounds; and water.
Statement 2. The composition according to Statement 1, wherein the one or more poloxamer compounds is selected from the group consisting of poloxamer 188, poloxamer 407, poloxamer 108, poloxamer 1107, poloxamer 338, poloxamer 101, poloxamer 105, poloxamer 122, poloxamer 123, poloxamer 124, poloxamer 181, poloxamer 182, poloxamer 183, poloxamer 184, poloxamer 185, poloxamer 212, poloxamer 215, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 401, poloxamer 402, poloxamer 403, poloxamer 105 benzoate, poloxamer 182 dibenzoate, and any combination thereof.
Statement 3. The composition according to Statement 1, wherein the one or more poloxamer compounds is selected from the group consisting of poloxamer 188, poloxamer 407, poloxamer 108, poloxamer 1107, poloxamer 338, and any combination thereof.
Statement 4. The composition according to Statement 1, wherein the one or more poloxamer compounds is selected from the group consisting of poloxamer 188, poloxamer 407, poloxamer 108, poloxamer 338, poloxamer 217, poloxamer 237, poloxamer 238, poloxamer 288, and any combination thereof.
Statement 5. The composition according to Statement 1, wherein the one or more poloxamer compounds comprises a mixture of poloxamer 188 and poloxamer 407.
Statement 6. The composition according to Statement 1, wherein the one or more poloxamer compounds comprises a mixture of poloxamer 188, poloxamer 407, and poloxamer 1107.
Statement 7. The composition according to Statement 1, wherein each of the one or more poloxamer compounds are characterized by an ethylene oxide (EO) content of at least 70%.
Statement 8. The composition according to Statement 1, comprising two or more poloxamer compounds selected from the group consisting of poloxamer 188, poloxamer 407, poloxamer 108, poloxamer 338, poloxamer 217, poloxamer 237, poloxamer 238, poloxamer 288, poloxamer 1107, and any combination thereof.
Statement 9. The composition according to Statement 1, comprising two or more poloxamer compounds selected from the group consisting of poloxamer 188, poloxamer 407, poloxamer 108, poloxamer 1107, poloxamer 338, and any combination thereof.
Statement 10. The composition according to Statement 8 or Statement 9, wherein the two or more poloxamer compounds comprises poloxamer 188.
Statement 11. The composition according to Statement 8 or Statement 9, wherein one of the two or more poloxamer compounds is poloxamer 188.
Statement 12. The composition according to Statement 1, wherein the one or more poloxamer compounds comprises poloxamer 188.
Statement 13. The composition according to any one of Statements 1-12, wherein the one or more poloxamer compounds comprises from about 0.5 weight % to about 65.0 weight %, or from about 25 weight % to about 45 weight %, or from about 25 weight % to about 55 weight %, or from about 5 weight % to about 15 weight %, or from about 5 weight % to about 18 weight %, or from about 7.5 weight % to about 12 weight % of the total weight of the composition.
Statement 14. The composition according to any one of Statements 1-12, wherein the one or more poloxamer compounds comprises from about 0.5 weight % to about 5.0 weight % or from about 1.0 weight % to about 4.0 weight % of the total weight of the composition.
Statement 15. The composition according to any one of Statements 1-12, wherein the one or more poloxamer compounds comprises from about 2.0 weight % to about 5.0 weight % of the total weight of the composition.
Statement 16. The composition according to any one of Statements 1-12, wherein the combined weight of the one or more poloxamer compounds comprises from about 0.5 weight % to about 65.0 weight %, or from about 25 weight % to about 45 weight %, or from about 25 weight % to about 55 weight %, or from about 5 weight % to about 15 weight %, or from about 5 weight % to about 18 weight %, or from about 7.5 weight % to about 12 weight % of the total weight of the composition.
Statement 17. The composition according to any one of Statements 1-12, wherein the combined weight of the one or more poloxamer compounds comprises from about 0.5 weight % to about 5.0 weight % or from about 1.0 weight % to about 4.0 weight % of the total weight of the composition.
Statement 18. The composition according to any one of Statements 1-12, wherein the combined weight of the one or more poloxamer compounds comprises from about 2.0 weight % to about 5.0 weight % of the total weight of the composition.
Statement 19. The composition according to any one of Statements 1-18, wherein poloxamer 188 comprises from about 0.5 weight % to about 65.0 weight %, or from about 25 weight % to about 45 weight %, or from about 25 weight % to about 55 weight %, or from about 5 weight % to about 15 weight %, or from about 5 weight % to about 18 weight %, or from about 7.5 weight % to about 12 weight % of the total weight of the composition.
Statement 20. The composition according to any one of Statements 1-18, wherein poloxamer 188 comprises from about 0.5 weight % to about 5.0 weight % or from about 1.0 weight % to about 4.0 weight % of the total weight of the composition.
Statement 21. The composition according to any one of Statements 1-18, wherein poloxamer 188 comprises from about 2.0 weight % to about 5.0 weight % of the total weight of the composition.
Statement 22. The composition according to any one of Statements 1-21, wherein PHMB comprises from about 0.01 weight % to about 0.5 weight % of the total weight of the composition.
Statement 23. The composition according to any one of Statements 1-21, wherein PHMB comprises from about 0.05 weight % to about 0.15 weight % of the total weight of the composition.
Statement 24. The composition according to any one of Statements 1-21, wherein PHMB comprises from about 0.04 weight % to about 0.08 weight % of the total weight of the composition.
Statement 25. The composition according to any one of Statements 1-21, wherein PHMB comprises about 0.1 weight % of the total weight of the composition.
Statement 26. The composition according to any one of Statements 1-25, further comprising benzalkonium chloride (BZK).
Statement 27. The composition according to Statement 26, wherein BZK comprises from about 0.01 weight % to about 0.5 weight % of the total weight of the composition.
Statement 28. The composition according to Statement 26, wherein BZK comprises from about 0.05 weight % to about 0.15 weight % of the total weight of the composition.
Statement 29. The composition according to Statement 26, wherein BZK comprises from about 0.04 weight % to about 0.08 weight % of the total weight of the composition.
Statement 30. The composition according to Statement 26, wherein BZK comprises 0.13 weight % of the total weight of the composition.
Statement 31. The composition according to any one of Statements 1-30, wherein the composition is a saline solution.
Statement 32. The composition according to any one of Statements 1-31, wherein the composition comprises from about 0.7 weight percent to about 1.5 weight percent sodium chloride (NaCl), based on the total weight of the composition.
Statement 33. The composition according to any one of Statements 1-31, wherein the composition comprises from about 0.8 weight percent to about 1.1 weight percent sodium chloride (NaCl), based on the total weight of the composition.
Statement 34. The composition according to any one of Statements 1-31, wherein the composition comprises from about 0.85 weight percent to about 0.95 weight percent sodium chloride (NaCl), based on the total weight of the composition.
Statement 35. The composition according to any one of Statements 1-31, wherein the composition comprises about 0.9 weight percent sodium chloride (NaCl), based on the total weight of the composition.
Statement 36. The composition according to any one of Statements 1-35, wherein the composition comprises at least 94 weight percent water, based on the total weight of the composition.
Statement 37. The composition according to any one of Statements 1-35, wherein the composition comprises at least 95 weight percent water, based on the total weight of the composition.
Statement 38. The composition according to any one of Statements 1-35, wherein the composition comprises at least 96 weight percent water, based on the total weight of the composition.
Statement 39. The composition according to any one of Statements 1-35, wherein the compositions comprises from about 94 weight percent to about 97 weight percent water, based on the total weight of the composition.
Statement 40. The composition according to Statement 1, comprising: PHMB; poloxamer 188; and water.
Statement 41. The composition according to Statement 1, comprising: PHMB; poloxamer 188; benzalkonium chloride (BZK); and water.
Statement 42. The composition according to Statement 1, comprising: PHMB; poloxamer 188; poloxamer 407; and water.
Statement 43. The composition according to Statement 1, comprising: PHMB; poloxamer 188; poloxamer 407; benzalkonium chloride (BZK); and water.
Statement 44. The composition according to any one of Statements 40-43, further comprising sodium chloride (NaCl).
Statement 45. The composition according to Statement 1, comprising: from about 0.05 to about 0.15 weight percent PHMB; from about 0.5 to about 5.0 weight percent poloxamer 188; and water.
Statement 46. The composition according to Statement 1, comprising: from about 0.05 to about 0.15 weight percent PHMB; from about 0.5 to about 65.0 weight percent poloxamer 188; from about 0.05 to about 0.15 weight percent benzalkonium chloride (BZK); and water.
Statement 47. The composition according to Statement 1, comprising: from about 0.05 to about 0.15 weight percent PHMB; from about 0.5 to about 15 weight percent poloxamer 188; from about 0.5 to about 2.5 weight percent poloxamer 407; and water.
Statement 48. The composition according to Statement 1, comprising: from about 0.05 to about 0.15 weight percent PHMB; from about 0.5 to about 2.5 weight percent poloxamer 188; from about 0.5 to about 2.5 weight percent poloxamer 407; from about 0.05 to about 0.15 weight percent benzalkonium chloride (BZK); and water.
Statement 49. The composition according to Statement 1, comprising: from about 0.05 to about 0.1 weight percent PHMB; from about 25 to about 45 weight percent poloxamer 188; and water.
Statement 50. The composition according to Statement 1, comprising: from about 0.05 to about 0.1 weight percent PHMB; from about 0.5 to about 2.0 weight percent poloxamer 188; from about 0.05 to about 0.13 weight percent benzalkonium chloride (BZK); and water.
Statement 51. The composition according to Statement 1, comprising: from about 0.05 to about 0.1 weight percent PHMB; from about 0.5 to about 2.0 weight percent poloxamer 188; from about 0.5 to about 2.0 weight percent poloxamer 407; and water.
Statement 52. The composition according to Statement 1, comprising: from about 0.05 to about 0.1 weight percent PHMB; from about 0.5 to about 2.0 weight percent poloxamer 188; from about 0.5 to about 2.0 weight percent poloxamer 407; from about 0.05 to about 0.13 weight percent benzalkonium chloride (BZK); and water.
Statement 53. The composition according to Statement 1, comprising: about 0.1 weight percent PHMB; from about 2.0 to about 4.0 weight percent poloxamer 188; and water.
Statement 54. The composition according to Statement 1, comprising: about 0.1 weight percent PHMB; from about 2.0 to about 4.0 weight percent poloxamer 188; about 0.13 weight percent benzalkonium chloride (BZK); and water.
Statement 55. The composition according to Statement 1, comprising: about 0.05 weight percent PHMB; from about 2.0 to about 4.0 weight percent poloxamer 188; about 0.05 weight percent benzalkonium chloride (BZK); and water.
Statement 56. The composition according to Statement 1, comprising: about 0.1 weight percent PHMB; about 2.0 weight percent poloxamer 188; about 2.0 weight percent poloxamer 407; and water.
Statement 57. The composition according to Statement 1, comprising: about 0.1 weight percent PHMB; about 1.0 weight percent poloxamer 188; about 1.0 weight percent poloxamer 407; and water.
Statement 58. The composition according to Statement 1, comprising: about 0.1 weight percent PHMB; about 2.0 weight percent poloxamer 188; about 2.0 weight percent poloxamer 407; about 0.13 weight percent benzalkonium chloride (BZK); and water.
Statement 59. The composition according to Statement 1, comprising: about 0.1 weight percent PHMB; about 1.0 weight percent poloxamer 188; about 1.0 weight percent poloxamer 407; about 0.13 weight percent benzalkonium chloride (BZK); and water.
Statement 60. The composition according to Statement 1, comprising: about 0.05 weight percent PHMB; about 2.0 weight percent poloxamer 188; about 2.0 weight percent poloxamer 407; about 0.05 weight percent benzalkonium chloride (BZK); and water.
Statement 61. The composition according to Statement 1, comprising: about 0.05 weight percent PHMB; about 1.0 weight percent poloxamer 188; about 1.0 weight percent poloxamer 407; about 0.05 weight percent benzalkonium chloride (BZK); and water.
Statement 62. The composition according to Statement 1, comprising: from about 0.05 to about 0.15 weight percent PHMB; from about 0.5 to about 2.5 weight percent poloxamer 188; from about 0.5 to about 2.5 weight percent one or more poloxamer compounds; and water.

SO
Statement 63. The composition according to Statement 1, comprising: from about 0.05 to about 0.15 weight percent PHMB; from about 0.5 to about 2.5 weight percent poloxamer 188; from about 0.5 to about 2.5 weight percent of one or more poloxamer compounds; from about 0.05 to about 0.15 weight percent benzalkonium chloride (BZK); and water.
Statement 64. The composition according to Statement 62 or Statement 63, wherein each of the one or more poloxamer compounds are characterized by an ethylene oxide (EO) content of at least 70%.
Statement 65. The composition according to Statement 62 or Statement 63, wherein the one or more poloxamer compounds is selected from the group consisting of poloxamer 407, poloxamer 108, poloxamer 1107, poloxamer 338, poloxamer 101, poloxamer 105, poloxamer 122, poloxamer 123, poloxamer 124, poloxamer 181, poloxamer 182, poloxamer 183, poloxamer 184, poloxamer 185, poloxamer 212, poloxamer 215, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 401, poloxamer 402, poloxamer 403, poloxamer 105 benzoate, poloxamer 182 dibenzoate, and any combination thereof.
Statement 66. The composition according to Statement 62 or Statement 63, wherein the one or more poloxamer compounds is selected from the group consisting of poloxamer 407, poloxamer 108, poloxamer 1107, poloxamer 338, and any combination thereof.
Statement 67. The composition according to Statement 62 or Statement 63, wherein the one or more poloxamer compounds is selected from the group consisting of poloxamer 407, poloxamer 108, poloxamer 338, poloxamer 217, poloxamer 237, poloxamer 238, poloxamer 288, and any combination thereof.
Statement 68. The composition according to Statement 62 or Statement 63, wherein the one or more poloxamer compounds comprises a mixture of poloxamer 407 and poloxamer 1107.
Statement 69. The composition according to Statement 62 or Statement 63, wherein the one or more poloxamer compounds is selected from the group consisting of poloxamer 407, poloxamer 108, poloxamer 338, poloxamer 217, poloxamer 237, poloxamer 238, poloxamer 288, poloxamer 1107, and any combination thereof.
Statement 70. The composition according to any one of Statements 45-69, further comprising from about 0.8 to about 1.0 weight present sodium chloride (NaCl), based on the total weight of the composition.
Statement 71. The composition according to any one of Statements 45-70, further comprising from about 93.7 weight percent to about 98.0 weight present water.
Statement 72. The composition according to any one of Statements 1 to 71, wherein the composition does not include betaine, trimethylglycine (TMG), or povidone-iodine.
Statement 73. The composition according to any one of Statements 1 to 71, wherein the composition excludes betaine, trimethylglycine (TMG), or povidone-iodine.
Statement 74. A method of irrigating a surgically-created implant pocket in a subject, the method comprising: (a) contacting the implant pocket with the composition according to any one of Statements 1-73.
Statement 75. The method according to Statement 74 or Statement 75, wherein contacting the implant pocket with the composition comprises causing an irrigation solution to pass through a dissection tunnel connecting the implant pocket to an incision on a skin of a subject such that it enters and at least partially fills the implant pocket, the irrigation solution comprising the composition according to any one of Statements 1-73.
Statement 76. The method according to Statement 74, wherein contacting the implant pocket with the composition comprises: rinsing the implant pocket with an irrigation solution, the irrigation solution comprising the composition according to any one of Statements 1-73.
Statement 77. The method according to Statement 75 or Statement 76, further comprising: (b) removing at least a portion of the irrigation solution from the implant pocket.
Statement 78. The method according to Statement 75 or Statement 76, further comprising: (b) substantially removing the irrigation solution from the implant pocket.
Statement 79. The method according to Statement 77 or Statement 78, wherein removing the irrigation solution, or at least a portion thereof, comprises: causing, via suction, the irrigation solution, or at least a portion thereof, to exit the implant pocket and pass through the dissection tunnel so as to be removed from the subject.
Statement 80. The method according to any one of Statements 77-79, further comprising: (c) repeating steps (a)-(b) two (2) to six (6) times.
Statement 81. The method according to any one of Statements 77-80, further comprising: (d) rinsing the implant pocket with a sterile saline solution.
Statement 82. The method according to any one of Statements 77-80, further comprising: (d) causing a sterile saline solution to pass through a dissection tunnel connecting the implant pocket to an incision on a skin of a subject such that it enters and at least partially fills the implant pocket.
Statement 83. The method according to Statement 81 or Statement 82, further comprising: causing, via suction, the sterile saline solution, or at least a portion thereof, to exit the implant pocket and pass through the dissection tunnel so as to be removed from the subject.
Statement 84. The method according to any one of Statements 74-83, wherein the surgically-created implant pocket is for the insertion of a prosthetic implant.
Statement 85. The method according to any one of Statements 74-83, wherein the surgically-created implant pocket is intended to contain a prosthetic implant.
Statement 86. The method according to any one of Statements 74-83, further comprising: (e) inserting a prosthetic implant into the surgically-created implant pocket.
Statement 87. The method according to any one of Statements 84-86, wherein the prosthetic implant is selected from the group consisting of filled implants or pre-filled implants, unfilled implants, saline implants, silicone gel implants, textured implants, smooth implants, highly cohesive silicone gel implants, oil-filled implants.
Statement 88. The method according to any one of Statements 84-86, wherein the prosthetic implant is selected from the group consisting of a breast implant, a gluteal implant, a buttock implant, and a facial implant.
Statement 89. The method according to any one of Statements 84-86, wherein the prosthetic implant is a silicone implant or a silicone gel implant selected from the group consisting of a silicone breast implant, a silicone gluteal implant, a silicone buttock implant, and a silicone facial implant.
Statement 90. The method according to any one of Statements 84-86, wherein the prosthetic implant is selected from the group consisting of an implantable device, a pacemaker, a joint replacement prosthesis, a tissue graft, an allograft, and an autograft.
Statement 91. The method according to any one of Statements 74-90, wherein step (a) occurs or is performed after a capsulectomy is performed on the implant pocket.
Statement 92. The method according to any one of Statements 74-90, wherein contacting the implant pocket with the composition occurs after a capsulectomy is performed on the implant pocket.
Statement 93. The method according to any one of Statements 74-90, wherein the surgically-created implant pocket is at least partially formed as a result of a capsulectomy.
Statement 94. A method of removing silicone gel or silicone residue from a surgically-created implant pocket in a subject, the method comprising: (a) contacting the implant pocket with the composition according to any one of Statements 1-73.
Statement 95. The method according to Statement 94, wherein the silicone gel or silicone residue is derived from or the result of a ruptured silicone gel implant.
Statement 96. A method of treating a subject having a surgically-created implant pocket that contains silicone gel or that has been exposed to silicone as a result of the rupture of a silicone gel implant, the method comprising: (a) contacting the implant pocket with the composition according to any one of Statements 1-73.
Statement 97. The method according to any one of Statements 94-96, wherein contacting the implant pocket with the composition comprises causing an irrigation solution to pass through a dissection tunnel connecting the implant pocket to an incision on a skin of a subject such that it enters and at least partially fills the implant pocket, the irrigation solution comprising the composition according to any one of Statements 1-73.
Statement 98. The method according to any one of Statements 94-96, wherein contacting the implant pocket with the composition comprises: rinsing the implant pocket with an irrigation solution, the irrigation solution comprising the composition according to any one of Statements 1-73.
Statement 99. The method according to any one of Statements 94-98, further comprising: (b) removing at least a portion of the irrigation solution from the implant pocket.
Statement 100. The method according to any one of Statements 94-98, further comprising: (b) substantially removing the irrigation solution from the implant pocket.
Statement 101. The method according to Statement 99 or Statement 100, wherein removing the irrigation solution, or at least a portion thereof, comprises: causing, via suction, the irrigation solution, or at least a portion thereof, to exit the implant pocket and pass through the dissection tunnel so as to be removed from the subject.
Statement 102. The method according to any one of Statements 99-101, further comprising: (c) repeating steps (a)-(b) two (2) to six (6) times.
Statement 103. The method according to any one of Statements 99-102, further comprising: (d) rinsing the implant pocket with a sterile saline solution.
Statement 104. The method according to any one of Statements 99-102, further comprising: (d) causing a sterile saline solution to pass through a dissection tunnel connecting the implant pocket to an incision on a skin of a subject such that it enters and at least partially fills the implant pocket.
Statement 105. The method according to Statement 103 or Statement 104, further comprising: causing, via suction, the sterile saline solution, or at least a portion thereof, to exit the implant pocket and pass through the dissection tunnel so as to be removed from the subject.
Statement 106. The method according to any one of Statements 94-105, further comprising removing a silicone gel implant or a shell thereof from the implant pocket.
Statement 107. The method according to Statement 106, wherein removing the silicone gel implant or a shell thereof from the implant pocket occurs or is performed prior to step (a) or contacting the implant pocket with the composition according to any one of Statements 1-73.
Statement 108. The method according to any one of Statements 94-105, further comprising: removing a silicone gel implant or a shell thereof from the implant pocket; and performing a capsulectomy on the implant pocket.
Statement 109. The method according to Statement 108, wherein removing the silicone gel implant or a shell thereof from the implant pocket and performing a capsulectomy on the implant pocket occurs or is performed prior to step (a) or contacting the implant pocket with the composition according to any one of Statements 1-73.
Statement 110. The method according to any one of Statements 94-109, further comprising: (e) inserting a new prosthetic implant into the surgically-created implant pocket.
Statement 111. The method according to Statement 110, wherein step (e) is performed after step (d).
Statement 112. The method according to Statement 110 or Statement 111, wherein the new prosthetic implant is selected from the group consisting of filled implants or pre-filled implants, unfilled implants, saline implants, silicone gel implants, textured implants, smooth implants, highly cohesive silicone gel implants, oil-filled implants.
Statement 113. The method according to Statement 110 or Statement 111, wherein the new prosthetic implant is selected from the group consisting of a breast implant, a gluteal implant, a buttock implant, and a facial implant.
Statement 114. The method according to any one of Statements 95-113, wherein the silicone gel implant is selected from the group consisting of a silicone gel breast implant, a silicone gel gluteal implant, a silicone gel buttock implant, and a silicone gel facial implant.
Statement 115. A method of irrigating a surgically-created implant pocket in a subject, the method comprising: (a) optionally, causing an incision in a skin of the subject; (b) optionally, generating an implant pocket and a dissection tunnel connecting the implant pocket to the incision in the skin of the subject; (c) contacting the implant pocket with an irrigation solution, wherein the irrigation solution is a composition according to any one of Statements 1-73; (d) causing, via suction, the irrigation solution, or at least a portion thereof, to exit the implant pocket and pass through the dissection tunnel so as to be removed from the implant pocket and the subject; (e) optionally, repeating steps (c)-(d) two (2) to six (6) times; (f) optionally, rinsing the implant pocket with a sterile saline solution; (g) optionally, inserting a prosthetic implant into the surgically-created implant pocket.
Statement 116. The method according to Statement 115, wherein contacting the implant pocket with the composition comprises causing an irrigation solution to pass through a dissection tunnel connecting the implant pocket to an incision on a skin of a subject such that it enters and at least partially fills the implant pocket.
Statement 117. The method according to Statement 115 or Statement 116, wherein rinsing the implant pocket with a sterile saline solution comprises: causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.
Statement 118. The method according to any one of Statements 115-117, wherein the prosthetic implant is selected from the group consisting of filled implants or pre-filled implants, unfilled implants, saline implants, silicone gel implants, textured implants, smooth implants, highly cohesive silicone gel implants, oil-filled implants.
Statement 119. The method according to any one of Statements 115-117, wherein the prosthetic implant is selected from the group consisting of a breast implants, a gluteal implants, buttock implants, and facial implants.
Statement 120. The method according to any one of Statements 115-117, wherein the prosthetic implant is a silicone implant or a silicone gel implant selected from the group consisting of silicone breast implants, silicone gluteal implants, silicone buttock implants, and silicone facial implants.
Statement 121. The method according to any one of Statements 115-117, wherein the prosthetic implant is selected from the group consisting of an implantable device, a pacemaker, a joint replacement prosthesis, a tissue graft, an allograft, and an autograft.
Statement 122. A method of removing silicone gel or silicone residue from a surgically-created implant pocket in a subject, the method comprising: (a) optionally, causing an incision in a skin of the subject; (b) optionally, generating a dissection tunnel connecting the implant pocket to the incision in the skin of the subject, the dissection tunnel comprising an aperture through which a ruptured silicone gel implant, or a portion thereof, may be removed; (c) optionally, removing a silicone gel implant or a shell thereof from the implant pocket; (d) optionally, removing, via mechanical agitation or suction, any loose silicone gel or debris from the implant pocket; (e) contacting the implant pocket with a silicone removal solution, the silicone removal solution comprising a composition according to any one of Statements 1-73; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with a silicone removal solution, the silicone removal solution comprising a composition according to any one of Statements 1-73; (k) optionally, causing, via suction, the silicone removal solution, or at least a portion thereof, to exit the implant pocket and pass through the dissection tunnel so as to be removed from the implant pocket and the subject; (1) optionally, repeating steps (j)-(k) two (2) to six (6) times; (m) optionally, rinsing the implant pocket with a sterile saline solution; (n) optionally, inserting a new prosthetic implant into the surgically-created implant pocket.
Statement 123. The method according to Statement 122, wherein the implant pocket contains silicone gel or silicone residue, the silicone gel or silicone residue derived from or the result of a ruptured silicone gel implant.
Statement 124. The method according to Statement 122 or Statement 123, wherein contacting the implant pocket with the silicone removal solution comprises causing the silicone removal solution to pass through a dissection tunnel connecting the implant pocket to an incision on a skin of a subject such that it enters and at least partially fills the implant pocket.
Statement 125. The method according to any one of Statements 122-124, wherein rinsing the implant pocket with a sterile saline solution comprises: causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.
Statement 126. The method according to any one of Statements 122-125, wherein the new prosthetic implant is selected from the group consisting of filled implants or pre-filled implants, unfilled implants, saline implants, silicone gel implants, textured implants, smooth implants, highly cohesive silicone gel implants, oil-filled implants.
Statement 127. The method according to any one of Statements 122-125, wherein the new prosthetic implant is selected from the group consisting of breast implants, gluteal implants, buttock implants, and facial implants.
Statement 128. The method according to any one of Statements 122-127, wherein the silicone gel implant is selected from the group consisting of silicone gel breast implants, silicone gel gluteal implants, silicone gel buttock implants, and silicone gel facial implants.
Statement 129. A method of treating a subject in need thereof, the subject having a surgically-created implant pocket that contains silicone gel or that has been exposed to silicone as a result of the rupture of a silicone gel implant, the method comprising: (a) optionally, causing an incision in a skin of the subject; (b) optionally, generating a dissection tunnel connecting the implant pocket to the incision in the skin of the subject, the dissection tunnel comprising an aperture through which a ruptured silicone gel implant may be removed; (c) optionally, removing a silicone gel implant or a shell thereof from the implant pocket; (d) optionally, removing, via mechanical agitation or suction, any loose silicone gel or debris from the implant pocket; (e) contacting the implant pocket with a silicone removal solution, the silicone removal solution comprising a composition according to any one of Statements 1-73; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with a silicone removal solution, the silicone removal solution comprising a composition according to any one of Statements 1-73; (k) optionally, causing, via suction, the silicone removal solution, or at least a portion thereof, to exit the implant pocket and pass through the dissection tunnel so as to be removed from the implant pocket and the subject; (l) optionally, repeating steps (j)-(k) two (2) to six (6) times; (m) optionally, rinsing the implant pocket with a sterile saline solution; (n) optionally, inserting a new prosthetic implant into the surgically-created implant pocket.
Statement 130. The method according to Statement 129, wherein contacting the implant pocket with the silicone removal solution comprises causing the silicone removal solution to pass through a dissection tunnel connecting the implant pocket to an incision on a skin of a subject such that it enters and at least partially fills the implant pocket.
Statement 131. The method according to Statement 129 or Statement 130, wherein rinsing the implant pocket with a sterile saline solution comprises: causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.
Statement 132. The method according to any one of Statements 129-131, wherein the new prosthetic implant is selected from the group consisting of filled implants or pre-filled implants, unfilled implants, saline implants, silicone gel implants, textured implants, smooth implants, highly cohesive silicone gel implants, oil-filled implants.
Statement 133. The method according to any one of Statements 129-131, wherein the new prosthetic implant is selected from the group consisting of breast implants, gluteal implants, buttock implants, and facial implants.
Statement 134. The method according to any one of Statements 129-133, wherein the silicone gel implant is selected from the group consisting of silicone gel breast implants, silicone gel gluteal implants, silicone gel buttock implants, and silicone gel facial implants.
Statement 135. A method of removing a ruptured silicone gel implant from a subject in need thereof, the method comprising: (a) optionally, causing an incision in a skin of the subject; (b) optionally, generating a dissection tunnel connecting an implant pocket in the subject to the incision in the skin of the subject, wherein the implant pocket contains a ruptured silicone gel implant and the dissection tunnel comprises an aperture through which a ruptured silicone gel implant may be removed; (c) optionally, removing the ruptured silicone gel implant or a shell thereof from the implant pocket; (d) optionally, removing, via mechanical agitation or suction, any loose silicone gel or debris from the implant pocket; (e) contacting the implant pocket with a silicone removal solution, the silicone removal solution comprising a composition according to any one of Statements 1-73; (f) causing, via suction, the silicone removal solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with a silicone removal solution, the silicone removal solution comprising a composition according to any one of Statements 1-73; (k) optionally, causing, via suction, the silicone removal solution, or at least a portion thereof, to exit the implant pocket and pass through the dissection tunnel so as to be removed from the implant pocket and the subject; (1) optionally, repeating steps (j)-(k) two (2) to six (6) times; (m) optionally, rinsing the implant pocket with a sterile saline solution; and (n) optionally, inserting a new prosthetic implant into the surgically-created implant pocket.
Statement 136. The method according to Statement 135, wherein contacting the implant pocket with the silicone removal solution comprises causing the silicone removal solution to pass through a dissection tunnel connecting the implant pocket to an incision on a skin of a subject such that it enters and at least partially fills the implant pocket.
Statement 137. The method according to Statement 135 or Statement 136, wherein rinsing the implant pocket with a sterile saline solution comprises: causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.
Statement 138. The method according to any one of Statements 135-137, wherein the new prosthetic implant is selected from the group consisting of filled implants or pre-filled implants, unfilled implants, saline implants, silicone gel implants, textured implants, smooth implants, highly cohesive silicone gel implants, oil-filled implants.
Statement 139. The method according to any one of Statements 135-137, wherein the new prosthetic implant is selected from the group consisting of breast implants, gluteal implants, buttock implants, and facial implants.
Statement 140. The method according to any one of Statements 135-139, wherein the silicone gel implant is selected from the group consisting of silicone gel breast implants, silicone gel gluteal implants, silicone gel buttock implants, and silicone gel facial implants.
Statement 141. A method of replacing a silicone gel implant in a subject in need thereof, the subject having a surgically-created implant pocket that contains a silicone gel implant, the method comprising: (a) optionally, causing an incision in a skin of the subject; (b) optionally, generating a dissection tunnel connecting the implant pocket to the incision in the skin of the subject, the dissection tunnel comprising an aperture through which the silicone gel implant may be removed; (c) optionally, removing the silicone gel implant or a shell thereof from the implant pocket; (d) optionally, removing, via mechanical agitation or suction, any loose silicone gel or debris from the implant pocket; (e) contacting the implant pocket with an irrigation solution, the irrigation solution comprising a composition according to any one of Statements 1-73; (f) causing, via suction, the irrigation solution, or at least a portion thereof, to exit the implant pocket and pass through the dissection tunnel so as to be removed from the implant pocket and the subject; (g) optionally, repeating steps (e)-(f) two (2) to six (6) times; (h) optionally, rinsing the implant pocket with a sterile saline solution; (i) optionally, performing a capsulectomy on the implant pocket; (j) optionally, contacting the implant pocket with an irrigation solution, the irrigation solution comprising a composition according to any one of Statements 1-73; (k) optionally, causing, via suction, the irrigation solution, or at least a portion thereof, to exit the implant pocket and pass through the dissection tunnel so as to be removed from the implant pocket and the subject; (1) optionally, repeating steps (c)-(d) two (2) to six (6) times; (m) optionally, rinsing the implant pocket with a sterile saline solution; (n) optionally, inserting a new prosthetic implant into the surgically-created implant pocket.
Statement 142. The method according to Statement 141, wherein the silicone gel implant in the implant pocket of the subject is intact.
Statement 143. The method according to Statement 141, wherein the silicone gel implant in the implant pocket of the subject is ruptured.
Statement 144. The method according to Statement 143, wherein the implant pocket contains silicone gel derived from the ruptured silicone gel implant.
Statement 145. The method according to any one of Statements 141-144, wherein contacting the implant pocket with the irrigation solution comprises causing the irrigation solution to pass through a dissection tunnel connecting the implant pocket to an incision on a skin of a subject such that it enters and at least partially fills the implant pocket.
Statement 146. The method according to any one of Statements 141-145, wherein rinsing the implant pocket with a sterile saline solution comprises: causing a sterile saline solution to pass through a dissection tunnel connecting the implant pocket to an incision on a skin of a subject such that it enters and at least partially fills the implant pocket; and causing, via suction, the sterile saline solution, or at least a portion thereof, to exit the implant pocket and pass through the dissection tunnel so as to be removed from the subject.
Statement 147. The method according to any one of Statements 141-146, wherein the new prosthetic implant is selected from the group consisting of filled implants or pre-filled implants, unfilled implants, saline implants, silicone gel implants, textured implants, smooth implants, highly cohesive silicone gel implants, oil-filled implants.
Statement 148. The method according to any one of Statements 141-146, wherein the new prosthetic implant is selected from the group consisting of breast implants, gluteal implants, buttock implants, and facial implants.
Statement 149. The method according to any one of Statements 141-148, wherein the silicone gel implant is selected from the group consisting of silicone gel breast implants, silicone gel gluteal implants, silicone gel buttock implants, and silicone gel facial implants.
Statement 150. The method according to any one of Statements 115-149, wherein the implant pocket is a breast implant pocket and the causing an incision in the skin of the subject may include an incision selected from the group consisting of an inframammary incision, a periareolar incision, an axillary incision, and a vertical incision (part of mastopexy pattern).

What is claimed is:

1. A composition for irrigating a surgically-created implant pocket in a subject, the composition comprising:
polyhexamethylene biguanide (PHMB);
one or more poloxamer compounds; and
water.

2. The composition according to claim 1, wherein the one or more poloxamer compounds is selected from the group consisting of poloxamer 188, poloxamer 407, poloxamer 108, poloxamer 1107, poloxamer 338, poloxamer 101, poloxamer 105, poloxamer 122, poloxamer 123, poloxamer 124, poloxamer 181, poloxamer 182, poloxamer 183, poloxamer 184, poloxamer 185, poloxamer 212, poloxamer 215, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 401, poloxamer 402, poloxamer 403, poloxamer 105 benzoate, poloxamer 182 dibenzoate, and any combination thereof.

3. The composition according to claim 1, wherein the one or more poloxamer compounds comprises a mixture of poloxamer 188 and poloxamer 407.

4. The composition according to claim 1, wherein each of the one or more poloxamer compounds are characterized by an ethylene oxide (EO) content of at least 70%.

5. The composition according to claim 1, wherein the combined weight of the one or more poloxamer compounds comprises from about 0.5 weight % to about 65.0 weight % of the total weight of the composition.

6. The composition according to claim 5, wherein poloxamer 188 comprises from about 0.5 weight % to about 5.0 weight % of the total weight of the composition.

7. The composition according to claim 5, wherein PHMB comprises from about 0.01 weight % to about 0.5 weight % of the total weight of the composition.

8. The composition according claim 7, further comprising benzalkonium chloride (BZK), wherein BZK comprises from about 0.01 weight % to about 0.5 weight % of the total weight of the composition.

9. The composition according to claim 8, wherein the composition comprises from about 0.7 weight percent to about 1.5 weight percent sodium chloride (NaCl), based on the total weight of the composition.

10. The composition according to claim 8, wherein the composition comprises at least 94 weight percent water, based on the total weight of the composition.

11. The composition according to claim 1, comprising:
from about 0.05 to about 0.15 weight percent PHMB;
from about 0.5 to about 5.0 weight percent poloxamer 188; and
water.

12. The composition according to claim 1, comprising:
from about 0.05 to about 0.15 weight percent PHMB;
from about 0.5 to about 15 weight percent poloxamer 188;
from about 0.5 to about 2.5 weight percent poloxamer 407; and
water.

13. The composition according to claim 1, comprising:
from about 0.05 to about 0.15 weight percent PHMB;
from about 0.5 to about 2.5 weight percent poloxamer 188;
from about 0.5 to about 2.5 weight percent one or more poloxamer compounds; and
water.

14. The composition according to claim 13, wherein each of the one or more poloxamer compounds are characterized by an ethylene oxide (EO) content of at least 70%.

15. The composition according to claim 1, wherein the composition excludes betaine, trimethylglycine (TMG), or povidone-iodine.

16. A method of irrigating a surgically-created implant pocket in a subject, the method comprising:
(a) contacting the implant pocket with a composition comprising:
polyhexamethylene biguanide (PHMB);
one or more poloxamer compounds; and
water;
wherein the surgically-created implant pocket is intended to contain a prosthetic implant; and
wherein contacting the implant pocket with the composition comprises causing the composition to pass through a dissection tunnel connecting the implant pocket to an incision on a skin of a subject such that the composition enters and at least partially fills the implant pocket.

17. The method according to claim 16, further comprising:
(b) removing at least a portion of the composition from the implant pocket;
(c) repeating steps (a)-(b) two (2) to six (6) times;
(d) rinsing the implant pocket with a sterile saline solution; and
(e) inserting a prosthetic implant into the surgically-created implant pocket, wherein the prosthetic implant is selected from the group consisting of filled implants or pre-filled implants, unfilled implants, saline implants, silicone gel implants, textured implants, smooth implants, highly cohesive silicone gel implants, oil-filled implants, a breast implant, a gluteal implant, a buttock implant, or a facial implant, an implantable device, a pacemaker, a joint replacement prosthesis, a tissue graft, an allograft, or an autograft.

18. A method of removing silicone gel or silicone residue from a surgically-created implant pocket in a subject, the method comprising:
(a) contacting the implant pocket with a composition comprising:
polyhexamethylene biguanide (PHMB);
one or more poloxamer compounds; and
water;
wherein the silicone gel or silicone residue is derived from or the result of a ruptured silicone gel implant.

19. The method according to claim 18, further comprising:
(b) removing at least a portion of the composition from the implant pocket;
(c) repeating steps (a)-(b) two (2) to six (6) times;
(d) rinsing the implant pocket with a sterile saline solution;
(e) removing a silicone gel implant or a shell thereof from the implant pocket;
(f) optionally, performing a capsulectomy on the implant pocket; and
(g) optionally, inserting a new prosthetic implant into the surgically-created implant pocket.

20. The method according to claim 19, wherein removing the silicone gel implant or a shell thereof from the implant pocket occurs or is performed prior to step (a) or prior to contacting the implant pocket with the composition.
